(19) United States
(12) Patent Application Publication
(10) Pub. No.: US 2026/0198872 A1
(43) Pub. Date: Jul. 16, 2026

(54) SYSTEMS AND METHODS FOR ATTENUATION CORRECTION

(71) Applicant: SHANGHAI UNITED IMAGING HEALTHCARE CO., LTD., Shanghai (CN)

(72) Inventors: Tiantian LI, Houston, TX (US); Liuchun HE, Shanghai (CN); Zhongzhi LIU, Wuhan (CN); Songsong TANG, Shanghai (CN); Xishan SUN, Houston, TX (US); Hongdi LI, Houston, TX (US)

(73) Assignee: SHANGHAI UNITED IMAGING HEALTHCARE CO., LTD., Shanghai (CN)

(21) Appl. No.: 19/562,742

(22) Filed: Mar. 10, 2026

Related U.S. Application Data
(63) Continuation-in-part of application No. 19/070,499, filed on Mar. 4, 2025.

(30) Foreign Application Priority Data
Mar. 13, 2024 (CN) 202410288352.5

(51) Int. Cl. A61B 6/03; A61B 6/00; G06T 12/10

(52) U.S. Cl. CPC A61B 6/037; A61B 6/5235; A61B 6/5282; G06T 12/10; G06T 2211/452

(57) ABSTRACT

A system may be provided. The system comprises an Emission Computed Tomography (ECT) scanner. The ECT scanner may comprise a detector defining an imaging field and photon emission device. The photon emission device may be disposed outside the imaging field and configured to emit photons before and during an ECT scan. At least a portion of the photons may enter the imaging field and strike the detector.

### CROSS-REFERENCE TO RELATED APPLICATION

This application is a continuation-in-part of U.S. patent application Ser. No. 19/070,499, filed on Mar. 4, 2025, which claims priority of Chinese Patent Application No. 202410288352.5, filed on Mar. 13, 2024, the contents of which are entirely incorporated herein by reference.

### TECHNICAL FIELD

The present disclosure relates to the technical field of positron emission tomography imaging, and in particular to methods, systems, and storage media for attenuation correction.

### BACKGROUND

Attenuation correction is important for positron emission tomography (PET). Attenuation correction is usually achieved by fusing PET data with computed tomography (CT) data. The CT data provides density information of a tissue, which is used to calculate attenuation factors of photons. These attenuation factors are then applied to the PET data to remove an attenuation effect of the photons in the tissue. However, an attenuation map (u-map) derived based on the CT data has some limitations (e.g., a high radiation dose, a metal artifact, a beam-hardening artifact, a truncation of a great patient, and a patient motion between PET/CT scan may affect the derivation of the attenuation map).

There is therefore a need for systems and methods for attenuation correction that provide an accurate attenuation correction result.

### SUMMARY

One or more embodiments of the present disclosure provide a method for attenuation correction. The method includes: obtaining radiological coincidence event data of radiation of a target object using an imaging device; obtaining transmission data related to the target object; and obtaining an attenuation-corrected radiological image by performing an attenuation correction and reconstruction based on the transmission data and the radiological coincidence event data. In some embodiments, the transmission data includes at least one of: backscattering coincidence event data of the target object or lutetium background event data of the imaging device.

One or more embodiments of the present disclosure provide a system for attenuation correction for implementing the method for attenuation correction. The system includes: a first obtaining module configured to obtain radiological coincidence event data of radiation of a target object using an imaging device; a second obtaining module configured to obtain transmission data related to the target object; and a reconstruction module configured to obtain an attenuation-corrected radiological image by performing an attenuation correction and reconstruction based on the transmission data and the radiological coincidence event data.

One or more embodiments of the present disclosure provide a non-transitory computer-readable storage medium storing computer instructions. When reading the computer instructions in the storage medium, a computer implements the method for attenuation correction.

One or more embodiments of the present disclosure provide a system. The system comprises an Emission Computed Tomography (ECT) scanner. The ECT scanner may comprise a detector defining an imaging field and photon emission device. The photon emission device may be disposed outside the imaging field and configured to emit photons before and during an ECT scan. At least a portion of the photons may enter the imaging field and strike the detector.

In some embodiments, the photon emission device is configured such that the photons irradiate a target region, and the target region covers the detector but does not cover a scanning object.

In some embodiments, the target region only covers the detector without covering any region outside the imaging field.

In some embodiments, the target region covers the detector and one or more regions outside the imaging field.

In some embodiments, an energy of the photons is in a range from 430 keV to 650 keV.

In some embodiments, the photon emission device comprises a photon emission source, a first photon shielding structure, a second photon shielding structure, and a fixing structure. The first photon shielding structure may be disposed on a first side of the photon emission source facing away from the detector. The second photon shielding structure may be disposed on a second side of the photon emission source facing the detector. The fixing structure secures positions of the photon emission source and the second photon shielding structure relative to the first photon shielding structure, the fixing structure being made of a material that allows the photons to pass through.

In some embodiments, the photon emission source includes at least one of a positron-emitting radionuclide or a single-photon-emitting radionuclide.

In some embodiments, the first photon shielding structure includes a concave structure, and the photon emission source is secured within the concave structure.

In some embodiments, the photon emission source is secured to the bottom surface of the concave structure.

In some embodiments, the photon emission source is secured to be suspended in the concave structure.

In some embodiments, the second photon shielding structure is secured outside or within the concave structure and does not contact the bottom surface of the concave structure, and the second photon shielding structure is spaced apart from the photon emission source.

In some embodiments, the fixing structure comprises a first component surrounding the photon emission source, the first component is connected between the photon emission source and the first photon shielding structure, and the first component is connected between the second photon shielding structure and the first photon shielding structure.

In some embodiments, the fixing structure further comprises a second component connected between the photon emission source and the second photon shielding structure.

In some embodiments, the first component and the second component have different hardnesses.

In some embodiments, the first component is made of hard plastic or glass, and the second component is made of foam plastic.

In some embodiments, the photon emission device is arranged at a central axis of the imaging field and spaced apart from the detector along the central axis.

In some embodiments, the ECT scanner includes a pair of photon emission devices symmetrically arranged relative to a central axis of the imaging field, and the pair of photon emission devices are spaced apart from the detector along the central axis.

In some embodiments, the system further comprises a processing device configured to perform operations including: obtaining first scan data collected by the detector in a blank scan without a scanning object in the imaging field; obtaining second scan data collected by the detector in the ECT scan with the scanning object in the imaging field; and generating an attenuation-corrected ECT image of the scanning object based on the first scan data and the second scan data.

In some embodiments, the generating an attenuation-corrected ECT image of the scanning object based on the first scan data and the second scan data comprises: determining first backscattering coincidence event data based on the first scan data; determining radiological coincidence event data and second backscattering coincidence event data based on the second scan data; and generating the attenuation-corrected ECT image of the scanning object based on the first backscattering coincidence event data, the second backscattering coincidence event data, and the radiological coincidence event data.

In some embodiments, the generating the attenuation-corrected ECT image of the scanning object based on the first backscattering coincidence event data, the second backscattering coincidence event data, and the radiological coincidence event data comprises: determining reference blank scanning estimation for backscattering corresponding to the photon emission device based on the first backscattering coincidence event data, a scan duration of the blank scan, and a scan duration of the ECT scan; and generating the attenuation-corrected ECT image of the scanning object based on the reference blank scanning estimation for backscattering, the second backscattering coincidence event data, and the radiological coincidence event data.

One or more embodiments of the present disclosure provide a method. The method may be implemented on a computing device having at least one storage device and at least one processor. The method may comprise obtaining first scan data collected by an Emission Computed Tomography (ECT) scanner in a blank scan without a scanning object in the imaging field. The ECT scanner comprises a detector and a photon emission device, the detector defining an imaging field and collecting scan data, the photon emission device being disposed outside the imaging field and configured to emit photons before and during an ECT scan, wherein at least a portion of the photons enter the imaging field and strike the detector. The method may also comprise obtaining second scan data collected by the detector in the ECT scan with the scanning object in the imaging field. The method may further comprise generating an attenuation-corrected ECT image of the scanning object based on the first scan data and the second scan data.

One or more embodiments of the present disclosure provide a system. The system comprises at least one storage device storing a set of instructions for Emission Computed Tomography (ECT) imaging and at least one processor configured to communicate with the at least one storage device. When executing the set of instructions, the at least one processor is configured to direct the system to perform the following operations. The system may obtain scan data of a scanning object collected by an ECT scanner. The system may determine radiological coincidence event data and triple backscattering coincidence event data based on the scan data. The triple backscattering coincidence event data relates to backscattering coincidence events defined by three single events. The system may further generate an attenuation-corrected ECT image of the scanning object based on the triple backscattering coincidence event data and the radiological coincidence event data.

In some embodiments, the three single events correspond to a first energy window, a second energy window, and a third energy window, respectively. The first energy window covers 340 kev, the second energy window covers 170 kev, and the third energy window covers 511 kev.

In some embodiments, to generate an attenuation-corrected ECT image of the scanning object, the system may perform the following operations. The system may determine random event estimation for the triple backscattering coincidence events based on the scan data, a first delay window, and a second delay window. The system may further generate the attenuation-corrected ECT image of the scanning object based on the triple backscattering coincidence event data, the radiological coincidence event data, and the random event estimation.

In some embodiments, to determine random event estimation for the triple backscattering coincidence events based on the scan data, a first delay window, and a second delay window, the system may perform the following operations. The system may determine first random event estimation, second random event estimation, and third random event estimation based on the scan data and the first delay window, wherein the first random event estimation relates to a first random photon corresponding to the first energy window, the second random event estimation relates to a second random photon corresponding to the second energy window, the third random event estimation relates to a third random photon corresponding to the third energy window. The system may determine fourth random event estimation based on the first delay window, the second delay window, and the scan data, wherein the fourth random event estimation relates to the first random photon, the second random photon, and the third random photon. The system may further determine the random event estimation based on the first random event estimation, the second random event estimation, the third random event estimation, and the fourth random event estimation.

In some embodiments, to determine the random event estimation based on the first random event estimation, the second random event estimation, the third random event estimation, and the fourth random event estimation, the system may perform the following operations. The system may determine a sum of the first random event estimation, the second random event estimation, and the third random event estimation. The system may further determine the random event estimation by subtracting twice the fourth random event estimation from the sum of the first random event estimation, the second random event estimation, and the third random event estimation.

In some embodiments, the first delay window, the second delay window, and a prompt window for determining the triple backscattering coincidence events are not overlapped.

In some embodiments, to generate an attenuation-corrected ECT image of the scanning object, the system may perform the following operations. The system may determine double backscattering coincidence event data relating to backscattering coincidence events defined by two single events. The system may further determine whether there is an unknown photon emission source outside an imaging field of the ECT scanner based on the double backscattering coincidence event data and the triple backscattering coincidence event data. In response to determining that there is no unknown photon emission source, The system may generate the attenuation-corrected ECT image of the scanning object based on the triple backscattering coincidence event data, the double backscattering coincidence event data, and the radiological coincidence event data.

In some embodiments, to determine whether there is an unknown photon emission source outside an imaging field of the ECT scanner, the system may perform the following operations. The system may generate a first attenuation-corrected ECT image of the scanning object based on the triple backscattering coincidence event data and the radiological coincidence event data. The system may generate a second attenuation-corrected ECT image of the scanning object based on the double backscattering coincidence event data and the radiological coincidence event data. The system may further determine whether there is an unknown photon emission source outside an imaging field of the ECT scanner based on a difference between the first attenuation-corrected ECT image and the second attenuation-corrected ECT image.

In some embodiments, to generate an attenuation-corrected ECT image of the scanning object, the system may perform the following operations. The system may determine double backscattering coincidence event data relating to backscattering coincidence events defined by two single events. The system may determine whether there is an unknown photon emission source outside an imaging field of the ECT scanner based on the double backscattering coincidence event data and the triple backscattering coincidence event data. In response to determining that there is an unknown photon emission source, the system may further generate the attenuation-corrected ECT image of the scanning object based on the triple backscattering coincidence event data and the radiological coincidence event data without using the double backscattering coincidence event data.

One or more embodiments of the present disclosure provide a method for ECT imaging. The method may be implemented on a computing device having at least one storage device and at least one processor. The method may comprise obtaining scan data of a scanning object collected by an ECT scanner. The method may comprise determine radiological coincidence event data and triple backscattering coincidence event data based on the scan data. The triple backscattering coincidence event data relates to backscattering coincidence events defined by three single events. The method may further comprise generating an attenuation-corrected ECT image of the scanning object based on the triple backscattering coincidence event data and the radiological coincidence event data.

One or more embodiments of the present disclosure provide a non-transitory computer readable medium. The non-transitory computer readable medium comprises at least one set of instructions for ECT imaging. When executed by one or more processors of a computing device, the at least one set of instructions causes the computing device to perform a method. The method may comprise obtaining scan data of a scanning object collected by an ECT scanner. The method may comprise determine radiological coincidence event data and triple backscattering coincidence event data based on the scan data. The triple backscattering coincidence event data relates to backscattering coincidence events defined by three single events. The method may further comprise generating an attenuation-corrected ECT image of the scanning object based on the triple backscattering coincidence event data and the radiological coincidence event data.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 is a schematic diagram illustrating an application scenario of a system for attenuation correction according to some embodiments of the present disclosure;

FIG. 2 is a block diagram illustrating an exemplary processing device 120 according to some embodiments of the present disclosure;

FIG. 3 is a flowchart illustrating an exemplary process for attenuation correction according to some embodiments of the present disclosure;

FIG. 4 is a schematic diagram illustrating an exemplary backscattering coincidence event according to some embodiments of the present disclosure;

FIG. 5 is a schematic diagram illustrating an exemplary lutetium background event according to some embodiments of the present disclosure;

FIG. 6 is a flowchart illustrating an exemplary process for obtaining an attenuation-corrected radiological image of a target object according to some embodiments of the present disclosure;

FIG. 7 is a schematic diagram illustrating an exemplary process for obtaining an attenuation-corrected radiological image by performing an iterative reconstruction based on backscattering coincidence event data of the target object according to some embodiments of the present disclosure;

FIG. 8 is a schematic diagram illustrating an exemplary process for obtaining an attenuation-corrected radiological image by performing an iterative reconstruction based on lutetium background event data according to some embodiments of the present disclosure;

FIG. 9 is a schematic diagram illustrating an exemplary process for obtaining an attenuation-corrected radiological image by performing an iterative reconstruction based on lutetium background event data and backscattering coincidence event data according to some embodiments of the present disclosure;

FIG. 10 is a schematic diagram illustrating an exemplary symmetry of a PET system according to some embodiments of the present disclosure;

FIG. 11 is a schematic diagram illustrating exemplary attenuation images according to some embodiments of the present disclosure.

FIG. 12 is a schematic diagram illustrating an exemplary ECT scanner 1200 according to some embodiments of the present disclosure;

FIGS. 13A-13E are cross-section views illustrating exemplary photon emission devices according to some embodiments of the present disclosure;

FIG. 13F is a schematic diagram illustrating a perspective view of an exemplary photon emission device according to some embodiments of the present disclosure;

FIG. 14 is a schematic diagram illustrating an exemplary ECT scanner according to some embodiments of the present disclosure;

FIG. 15 is a schematic diagram illustrating an exemplary ECT scanner according to some embodiments of the present disclosure;

FIG. 16 is a schematic diagram illustrating an exemplary ECT scanner according to some embodiments of the present disclosure;

FIG. 17 is a flowchart illustrating an exemplary process for attenuation correction according to some embodiments of the present disclosure;

FIG. 18 is a schematic diagram illustrating an exemplary process for obtaining an attenuation-corrected ECT image according to some embodiments of the present disclosure;

FIG. 19 is a schematic diagram illustrating exemplary backscattering coincidence events generated by different photon emission sources according to some embodiments of the present disclosure;

FIG. 20 is a flowchart illustrating an exemplary process for attenuation correction based on a triple-coincidence strategy according to some embodiments of the present disclosure;

FIG. 21 is a schematic diagram illustrating an exemplary process for determining random event estimation for triple backscattering coincidence events according to some embodiments of the present disclosure; and

FIG. 22 is a flowchart illustrating an exemplary process for attenuation correction according to some embodiments of the present disclosure.

### DETAILED DESCRIPTION

To more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for those skilled in the art to apply the present disclosure to other similar scenarios in accordance with these drawings without creative labor. Unless obviously obtained from the context or the context illustrates otherwise, the same numeral in the drawings refers to the same structure or operation.

It should be understood that the terms “system,” “device,” “unit” and/or “module” as used herein is a way to distinguish between different components, elements, parts, sections or assemblies at different levels. However, the words may be replaced by other expressions if other words accomplish the same purpose.

As shown in the present disclosure and in the claims, unless the context clearly suggests an exception, the words “a,” “one,” “an” and/or “the” do not refer specifically to the singular, but may also include the plural. Generally, the terms “including” and “comprising” suggest only the inclusion of clearly identified operations and elements that do not constitute an exclusive list, and the method or device may also include other operations or elements.

Flowcharts are used in the present disclosure to illustrate operations performed by a system in accordance with embodiments of the present disclosure. It should be appreciated that the preceding or following operations are not necessarily performed in an exact sequence. Instead, the operations can be processed in reverse order or simultaneously. Also, it is possible to add other operations to these processes or remove an operation or operations from them.

An attenuation correction (AC) is an important operation in positron emission tomography (PET) imaging. During PET imaging, emitted positrons interact with surrounding tissues and generate 511 keV photons, which are subject to different degrees of attenuation as the photons pass through the tissues of a body. Therefore, correction on these attenuations is required to obtain a more accurate PET image.

In PET/computed tomography (CT) imaging, the attenuation correction is usually achieved by fusing PET data with CT data. The CT data provides information about density of the tissue, which is used to calculate attenuation factors of the photons. These attenuation factors are then applied to the PET data to remove an attenuation effect of the photons in the tissue.

However, an attenuation map (u-map) derived based on the CT data has some limitations. For example, a high radiation dose, a metal artifact, a beam-hardening artifact, a truncation of a great patient, and a patient motion between PET/CT scan may affect the derivation of the attenuation map.

The prior art performs the attenuation correction by jointly reconstructing an attenuation map and an activity map, and this allows for a direct extraction of attenuation factor information directly from radiological data. A maximum likelihood estimation (MLAA) of the attenuation map and the activity map is first calculated, and the PET and an attenuation image reconstruction are iterated alternatively in sequence using a maximum likelihood expectation maximization (MLEM) and a transmission tomography maximum likelihood (MLTR) algorithm. Similar to the MLAA, some studies propose the maximum likelihood estimation of the activity map and the attenuation correction factor (MLACF), where the attenuation factor is estimated after each update of the activity image or at each update of the activity image.

A potential problem with such algorithms is that, on the one hand, the MLAA or the MLACF may converge to a local optimum solution if initial values are not good enough. On the other hand, the MLAA and the MLACF use the same set of radiological data for estimating the activity map and the attenuation map, a crosstalk problem may also affect the speed of convergence and the quality of the estimated images.

To address the above issues, external rotated sources are used in some studies to reconstruct the attenuation map used directly for the attenuation correction or as the initial values of the attenuation map in the MLAA. A disadvantage of external source scanning is that an additional device is required, which increases the complexity of system design and user operation while also increasing a radiation dose received by a patient.

In some embodiments of the present disclosure, a method for attenuation correction is provided. In the method, an attenuation-corrected radiological image of a target object is reconstructed by obtaining transmission data, and by obtaining radiological coincidence event data of the target object. The transmission data includes backscattering coincidence event data of the target object. Without an aid of the CT data, the attenuation image or the attenuation factors are estimated and corrected without increasing the complexity of the system, a scanning time, or the radiation dose to the patient.

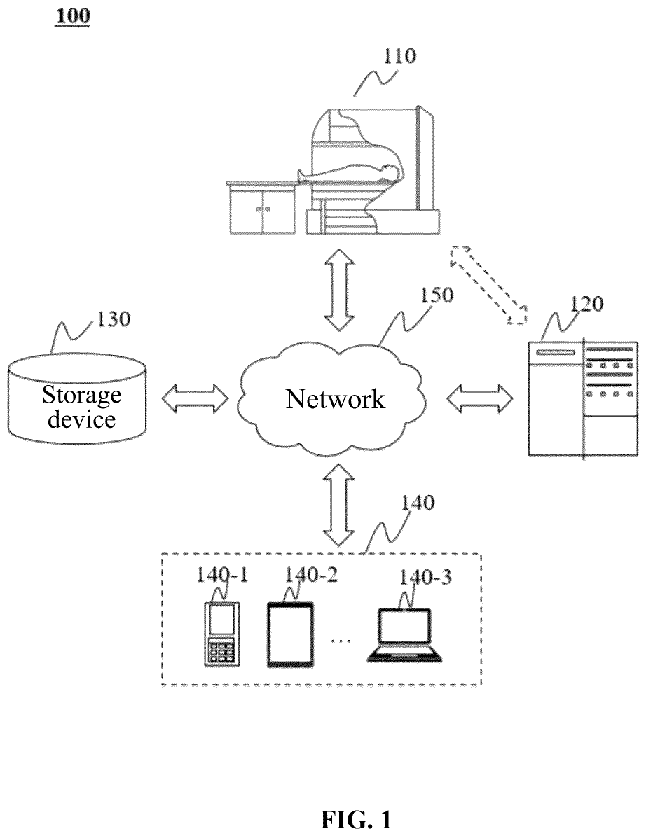

FIG. 1 is a schematic diagram illustrating an application scenario of a system for attenuation correction according to some embodiments of the present disclosure.

As shown in FIG. 1, in some embodiments, an application scenario of the system 100 for attenuation correction includes a scanning device 110, a processing device 120, a storage device 130, a terminal 140, and/or a network 150.

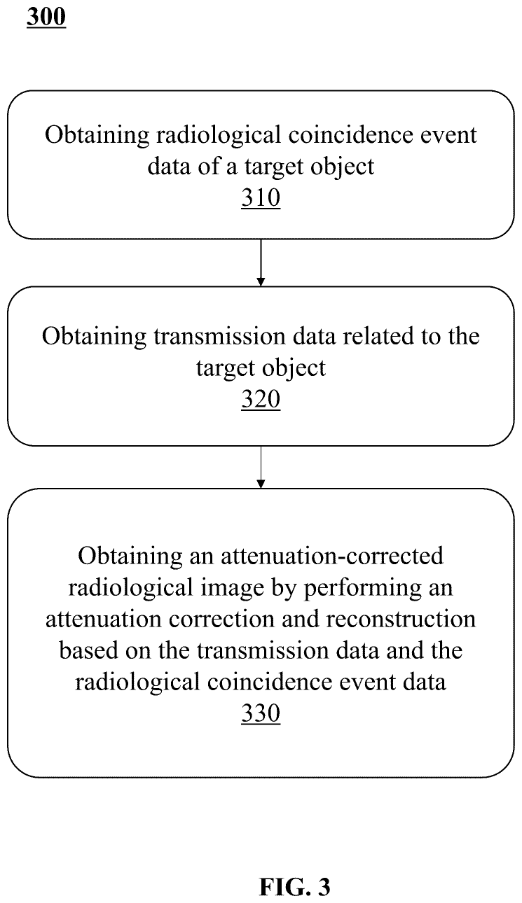

The scanning device 110 refers to a medical device that reproduces an internal structure of an object (e.g., a human body) as an image. In some embodiments, the scanning device 110 can be any medical device that images or treats a designated part of an object by means of radionuclides, for example, an emission computed tomography (ECT) scanner (e.g., a PET scanner, a single photon emission computed tomography (SPECT), etc.), a CT scanner, a PET-CT scanner, etc. The scanning device 110 provided above is for illustrative purposes only and is not a limitation of the scope of the present disclosure. A detector in the scanning device 110 may receive radiation from a radiation source and meter a received radiation. The detector includes a plurality of detector units arranged in one or more rings. In some embodiments, the scanning device 110 sends data and information related to the detector, such as an energy value of radiated photons received by the detector, an output value of the detector, etc., to the processing device 120. In some embodiments, the scanning device 110 collects transmission data, radiological coincidence event data of the scanning target object, etc., and sends them to the processing device 120. More descriptions about the transmission data, the target object, and the radiological coincidence event data may be found in FIG. 3 and the related descriptions. In some embodiments, the scanning device 110 receives an instruction, etc., sent by a physician through the terminal 140, and performs relevant operations, e.g., a radiation imaging, etc., according to the instruction. In some embodiments, the scanning device 110 exchanges data and/or information with other components of the system 100 (e.g., the processing device 120, the storage device 130, the terminal 140) through the network 150. In some embodiments, the scanning device 110 is directly connected to other components in the application scenario 100 of the system for attenuation correction. In some embodiments, the one or more components of the application scenario 100 of the system for attenuation correction (e.g., the processing device 120, the storage device 130) are included within the scanning device 110.

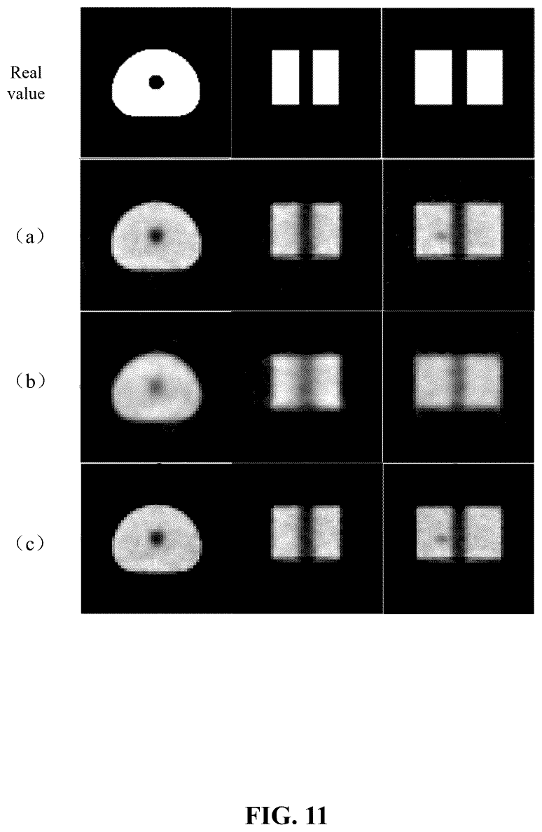

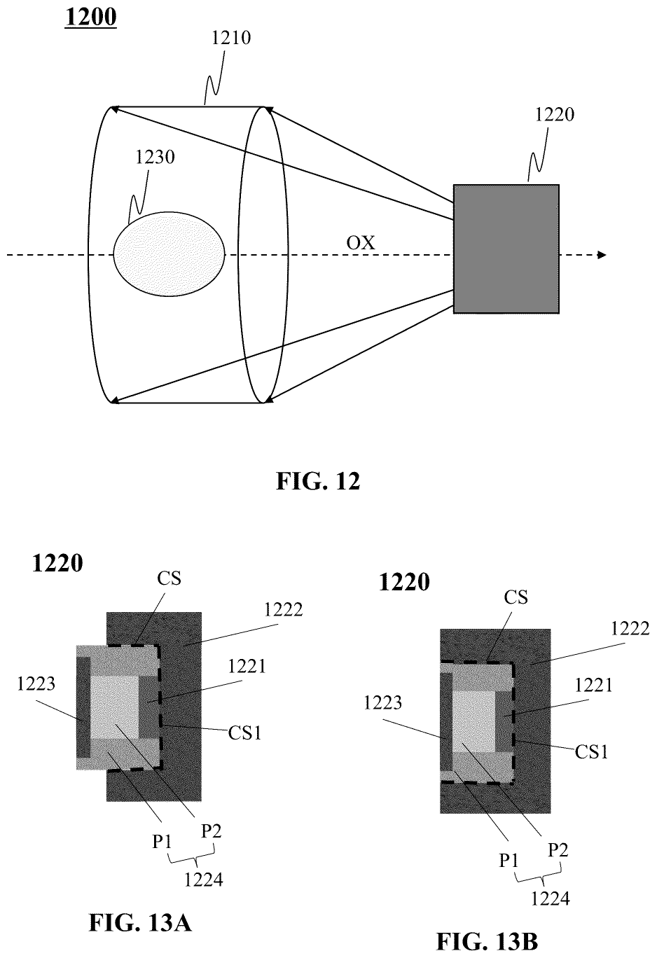

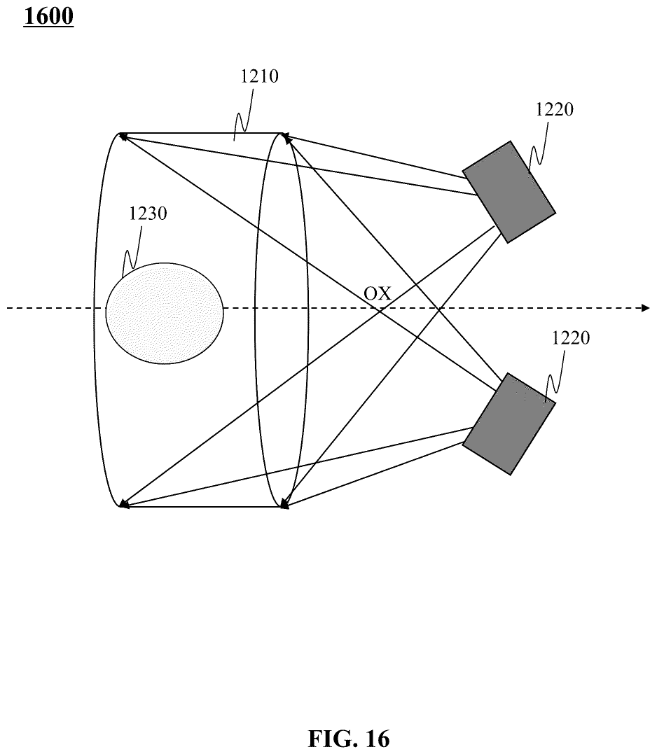

In some embodiments, the scanning device 110 is an ECT scanner, which includes a detector defining an imaging field and a photon emission device. The photon emission device is disposed outside the imaging field and configured to emit photons before and during an ECT scan, and at least a portion of the photons enter the imaging field and strike the detector. More descriptions about the ECT scanner may be found in FIG. 12 and FIG. 16, and their related descriptions.

The processing device 120 may process the data and/or information obtained from other devices or components of the system, and based on the data, the information, and/or a processing result, the processing device 120 may perform operations for correcting a scanning image as illustrated in some embodiments of the present disclosure, so as to accomplish one or more functions described in some embodiments of the present disclosure. For example, the processing device 120 obtains an attenuation-corrected radiological image by performing an attenuation correction and reconstruction based on the transmission data and the radiological coincidence event data. In some embodiments, the processing device 120 obtains pre-stored data and/or information, e.g., the transmittance data, the radiological coincidence event data, various calculation formulas, etc., from the storage device 130 for performing the method for attenuation correction according to some embodiments of the present disclosure.

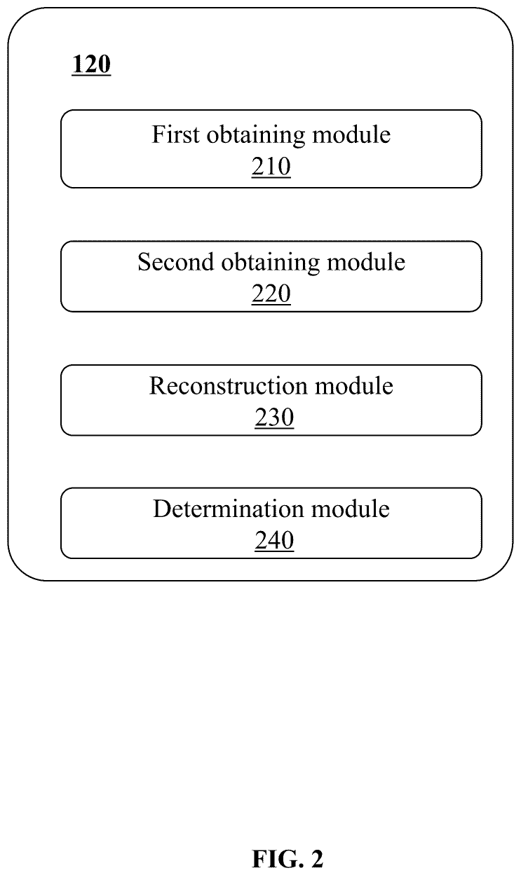

In some embodiments, the processing device 120 (e.g., one or more modules illustrated in FIG. 2) may execute instructions and may accordingly be directed to perform one or more processes (e.g., processes 300, 600, 1700, 2000, and 2200) described in the present disclosure. For example, each of the one or more processes may be stored in a storage device (e.g., the storage device 130) as a form of instructions, and invoked and/or executed by the processing device 120.

In some embodiments, the processing device 120 may be a single server or a server group. In some embodiments, the processing device 120 may be local to or remote from the system 100. Merely for illustration, only one processing device 120 is described in the system 100. However, it should be noted that the system 100 in the present disclosure may also include multiple processing devices. Thus operations and/or method steps that are performed by one processing device 120 as described in the present disclosure may also be jointly or separately performed by the multiple processing devices. For example, if in the present disclosure the processing device 120 of the system 100 executes both process A and process B, it should be understood that the process A and the process B may also be performed by two or more different processing devices jointly or separately in the system 100 (e.g., a first processing device executes process A and a second processing device executes process B, or the first and second processing devices jointly execute processes A and B).

In some embodiments, the processing device 120 includes one or more sub-processing devices (e.g., a single-core processing device or a multi-core processing device). Merely by way of example, the processing device 120 includes a central processing unit (CPU), an application-specific integrated circuit (ASIC), a graphics processor (GPU), a digital signal processor (DSP), a field programmable gate array (FPGA), a microcontroller unit (MCU), a reduced instruction set computer (RISC), a microprocessor, etc., or any combination of the above.

The storage device 130 stores data or information generated by other devices. In some embodiments, the storage device 130 stores the data and/or information collected by the scanning device 110, for example, the transmission data, the radiological coincidence event data, etc. The storage device 130 may include one or more storage components, each of which is an independent device or a part of other devices. The storage device may be local or be implemented through a cloud. In some embodiments, one or more components of the system 100 (e.g., the scanning device 110, the processing device 120, the terminal 140) include their own storage components.

The terminal 140 may control an operation of the scanning device 110. A physician may issue an operational instruction to the scanning device 110 through the terminal 140 to enable the scanning device 110 to complete a specified operation, such as irradiating a specified body part of the patient for imaging. In some embodiments, the terminal 140 is instructed to enable the processing device 120 to perform the method for attenuation correction according to some embodiments of the present disclosure. In some embodiments, the terminal 140 receives the attenuation-corrected radiological image, etc., from the processing device 120, so that the physician accurately determines a situation of the patient for an effective and targeted examination and/or treatment. In some embodiments, the terminal 140 is a mobile device 140-1, a tablet computer 140-2, a laptop computer 140-3, a desktop computer, and other input and/or output devices, or any combination thereof.

The network 150 may connect components of the system and/or connect the system to external resource portions. The network 150 may enable communications between components and other portions outside the system, so as to facilitate the exchange of the data and/or information. In some embodiments, the one or more components of the system 100 (e.g., the scanning device 110, the processing device 120, the storage device 130, the terminal 140) send the data and/or information to other components through the network 150. In some embodiments, the network 150 is any one or more of a wired network or a wireless network.

It should be noted that the foregoing description is provided for illustrative purposes only and is not intended to limit the scope of the present disclosure. For those skilled in the art, a wide variety of changes and modifications may be made under the guidance of the contents of the present disclosure. Features, structures, methods, and other characteristics of the exemplary embodiments described herein may be combined in various ways to obtain additional and/or alternative exemplary embodiments. For example, the processing device 120 is based on a cloud calculation platform, such as a public cloud, a private cloud, a community, and a hybrid cloud, etc. However, these changes and modifications do not depart from the scope of the present disclosure.

FIG. 2 is a block diagram illustrating an exemplary system processing device 120 according to some embodiments of the present disclosure.

In some embodiments, the processing device 120 includes a first obtaining module 210, a second obtaining module 220, and a reconstruction module 230. In some embodiments, the processing device 120 may further include a determination module 240.

In some embodiments, the first obtaining module 210 is configured to obtain radiological coincidence event data of a target object.

In some embodiments, the second obtaining module 220 is configured to obtain transmission data.

In some embodiments, the reconstruction module 230 is configured to obtain an attenuation-corrected radiological image by performing an attenuation correction and reconstruction based on the transmission data and the radiological coincidence event data. The transmission data includes backscattering coincidence event data of the target object.

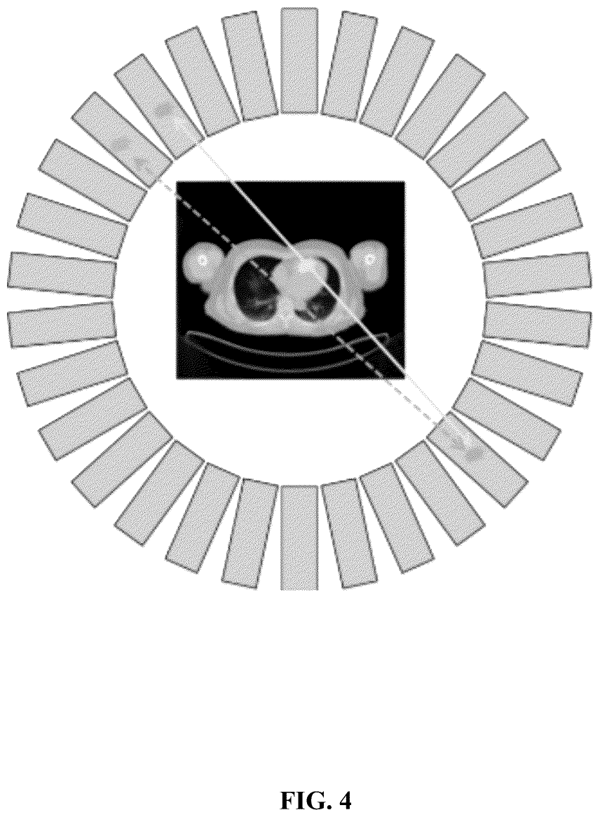

More descriptions about the transmission data, the target object, the radiological coincidence event data, the radiological image, the attenuation correction, and the backscattering coincidence event data may be found in FIG. 3, FIG. 4, and their related

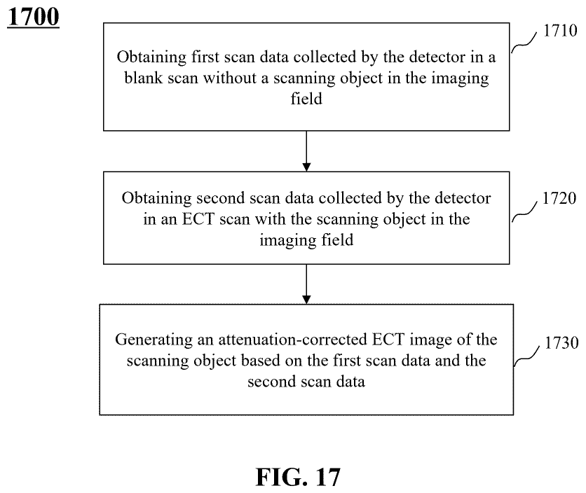

In some embodiments, the first obtaining module 210 may be configured to obtain first scan data collected by the detector in a blank scan without a scanning object in the imaging field. In some embodiments, the first obtaining module 210 and the second obtaining module 220 may be configured to obtain second scan data collected by the detector (i.e., the detector 1210) in an ECT scan with the scanning object in the imaging field. In some embodiments, the reconstruction module 230 may be configured to generate an attenuation-corrected ECT image of the scanning object based on the first scan data and the second scan data. More descriptions about the first scan data, the second scan data, and the generation of the attenuation-corrected ECT image may be found in FIG. 17 and their related descriptions.

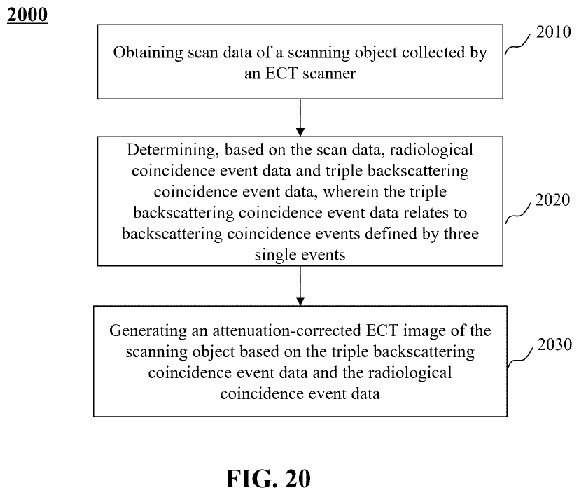

In some embodiments, the first obtaining module 210 may be configured to obtain scan data of a scanning object collected by an ECT scanner. In some embodiments, the determination module 240 may be configured to determine, based on the scan data, radiological coincidence event data and triple backscattering coincidence event data. In some embodiments, the reconstruction module 230 may be configured to generate an attenuation-corrected ECT image of the scanning object based on the triple backscattering coincidence event data and the radiological coincidence event data. More descriptions about the scan data, the determination of the radiological coincidence event data and the triple backscattering coincidence event data, and the generation of the attenuation-corrected ECT image may be found in FIG. 20 and their related descriptions.

It is noted that the above description of the system for attenuation correction and the modules thereof are for descriptive convenience only, and does not limit the present disclosure to the scope of the cited embodiments. It is to be understood that for those skilled in the art, after understanding the principle of the system, it may be possible to arbitrarily combine the individual modules or form a sub-system to be connected to the other modules without departing from this principle. In some embodiments, the first obtaining module 210, the second obtaining module 220, and the reconstruction module 230 in FIG. 2 are different modules in a single system, or a single module realizing the functions of two or more of the above-mentioned modules. For example, the individual modules share a common storage module, or the individual modules each has a respective storage module. Deformations such as these are within the scope of protection of the present disclosure.

FIG. 3 is a flowchart illustrating an exemplary process for attenuation correction according to some embodiments of the present disclosure. As shown in FIG. 3, a process 300 includes one or more of the following operations. In some embodiments, the process 300 is performed by the processing device 120.

In 310, radiological coincidence event data of a target object may be obtained. In some embodiments, operation 310 is performed by the processing device 120 or the first obtaining module 210.

The radiological coincidence event data refers to the data related to the radiological coincidence event. For example, the data related to the radiological coincidence event may include a count, a trajectory, etc., of the radiological coincidence events of the target object. The radiological coincidence events are also known as positive and negative electron annihilation coincidence events. A tracer needs to be introduced into the target subject before PET scanning is performed. The tracer emits positrons during the PET scanning. A great count of negatively charged electrons are naturally present in the target object, the positrons have the same mass and opposite charge as the electrons, and annihilation occurs when the positron collides with the electron (also known as an “annihilation event” or a “coincidence event”). The annihilation produces gamma photons (or radiation rays) of 511 keV energy in two opposite directions. A line connecting two y-photons may be called a line of response (LOR).

In some embodiments, the processing device 120 obtains the radiological coincidence event data for the target object through the scanning device 110.

In some embodiments, the processing device 120 obtains single event data of the target object; determines whether an energy and an arrival time of the single event data coincide to a second preset rule; and in response to the single event data coincides to the second preset rule, the processing device 120 determines the single event data that coincides with the second preset rule as the radiological coincidence event data. More descriptions about the target object, the single event data, the energy, and the arrival time may be found in the foregoing related descriptions.

The second preset rule may be set based on experience or demands. In some embodiments, the second preset rule includes: if the energies of two different single events in the single event data are both within a third preset energy window, and a difference between the arrival times of the two different single events is within a second preset time window; then data corresponding to the two different single events is the radiological coincidence event data.

The third preset energy window refers to an energy interval used to determine whether two single events are radiological coincidence events.

The second preset time window refers to a preset time interval used to distinguish whether a single event is a radiological coincidence event.

In some embodiments, the third preset energy window and the second preset time window are determined in a variety of manners. For example, the third preset energy window and the second preset time window are preset by experience or demands. Also, for example, the third preset energy window and the second preset time window are set based on one or more scanning device parameters. The scanning device parameter refers to data information related to the scanning device, for example, an axial length, an energy resolution, etc., of the scanning device. The processing device 120 may obtain the third preset energy window and the second preset time window by checking a table. The table includes different scanning device parameters and their corresponding third preset energy windows and second preset time windows. The table may be obtained by preset, historical data, etc.

In some embodiments, the processing device 120 presets parameters (the third preset energy window, the second preset time window, etc.) in the second preset rule, and determine whether the single event data complies with the second preset rule, and if the single event data complies with the second preset rule, the processing device 120 determines the single event data as the radiological coincidence event data.

For example, the processing device 120 sets the third preset energy window to be 425 keV-800 keV and the second preset time window to be 4.4 ns based on a PET scanning device, etc., and selects two different single events in the single event data. A single event 6 occurs before a single event 5; an energy of the single event 5 and an energy of the single event 6 are compared with the third preset energy window, and a difference between arrival times of the single event 5 and the single event 6 is compared with the second preset time window. If the energy of the single event 5 and the energy of the single event 6 are located in the third preset energy window, and the difference between the arrival times of the single event 5 and the single event 6 is located in the second preset time window, then the processing device 120 determines that the data corresponding to the single event 5 and the data corresponding to the single event 6 belongs to the radiological coincidence event data.

In some embodiments, the single event data is collected simultaneously, screened through different preset energy windows and preset time windows, and then the transmission data and the radiological coincidence event data are obtained. In some embodiments, the scanning device 110 uses an extended energy window for collecting the single events. It is understood that the extended energy window has a greater window and is capable of detecting a plurality of types of events (e.g., lutetium background radiation events, object scattering events, inter-crystal scattering events, etc.). Parameters of the extended energy window may be set based on experience or demands, e.g., the parameters of the extended energy window may include a low level discriminator (LLD): 100 keV; a high level discriminator (ULD): 1024 keV.

In some embodiments of the present disclosure, by obtaining the single event data of the target object, and determining the single event data that satisfies the second preset rule as the radiological coincidence event data, the single event data is comprehensively screened from the dimensions of the energy and the arrival time, so as to exclude a noise and interfering signals, and to improve an accuracy and a reliability of the screening of the radiological coincidence event data.

In 320, obtaining transmission data related to the target object may be obtained. In some embodiments, operation 320 is performed by the processing device 120 or the second obtaining module 220.

The transmission data refers to data that transmits the target object. The target object refers to an object to be scanned, for example, a living organism, a mold body, etc. The living organism may be a human body or an animal, etc., and the mold body may be a mold body of a variety of materials and shapes, for example, a water mold, a gel material mold body, a cylinder, a cuboid, etc. In some embodiments, the transmission data includes at least one of the following: backscattering coincidence event data of the target object or lutetium background event data of the imaging device.

The backscattering coincidence event data refers to data related to backscattering coincidence events. For example, a count, a trajectory, etc., of the backscattering coincidence events. A backscattering coincidence event refers to an event where two scattered photons are detected by different detector units of the detector within a coincidence time window corresponding to backscattering coincidence event detection.

In a PET detector, two 511 keV γ-rays interact with matter to produce a photoelectric effect and Compton scattering. The photoelectric effect refers that the y-rays interact with electrons in the matter and transfers all of their energy to an electron, which breaks away from atom. The Compton scattering refers that the γ-rays interacts with the electrons in the matter, but only a part of the energy is transferred to the electron, and the γ-rays change direction.

In the present disclosure, attention is given to a physical process by which the y rays undergo the Compton scattering in the PET detector. One or both of the γ-rays undergo the Compton scattering in a first crystal (shown by the dashed line in FIG. 4), with some energy transferred to the electrons, and the γ-rays undergo a backscattering (the backscattering refers to a phenomenon of waves, particles, or signals reflecting back from a direction from which they came), and penetrate back into the target object. Eventually, this γ-ray is detected by another crystal on the other side (as shown by the solid line in FIG. 4). After detection, the backscattering event forms a transmission LOR for a transmission image estimation.

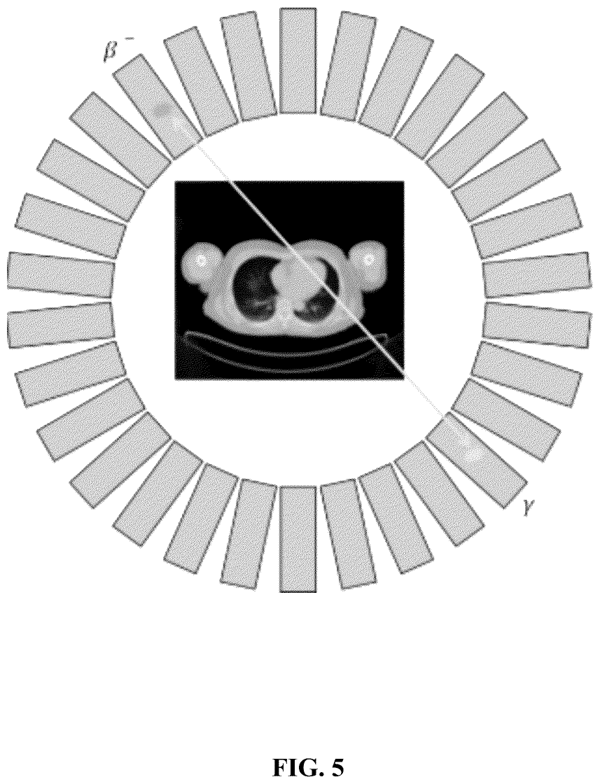

The lutetium background event data refers to the coincidence event data generated by a lutetium spontaneous background radiation of the crystals in a scanning device. For example, a count, a trajectory, etc., of the lutetium background event. As shown in FIG. 5, a common PET system uses a lutetium oxyorthosilicate (LSO) or a (LYSO) crystal as scintillation crystals containing isotope Lu-176, which is capable of generating the spontaneous background radiation.

In some embodiments, the processing device 120 obtains the transmission data through the scanning device 110. For example, the processing device 120 collects a background radiation signal from the scanning device to obtain the lutetium background event data. For another example, the processing device 120 scans the target object by the scanning device 110 to obtain the backscattering coincidence event data.

In some embodiments, the processing device 120 obtains the single event data of the target object; determine whether the energy and the arrival time of the single event data conforms to the first preset rule; and in response to the single event data conforming to the first preset rule, determine that the conforming to the first preset rule, determine that the single event data that meets the first preset rule is transmission data.

The single event data refers to data related to a single event. For example, the single event data includes counts, trajectories, etc., of a plurality of single events. The processing device 120 may obtain the single event data by scanning through the scanning device 110. The arrival time refers to a time when the detector detects the single event. The processing device 120 may scan the energy and the arrival time of the single event data obtained by the scanning device 110.

The first preset rule may be set based on experience or demands. In some embodiments, the first preset rule includes: if two different single events in the single event data have energies that are within a first preset energy window and a second preset energy window, respectively, and a difference between the arrival times of the two different single events is within the first preset time window; then the data corresponding to the two different single events is the transmission data.

Understandably, the energy and the arrival time of the transmission data have certain features, and in a case of the backscattering coincidence event data, a backscattering coincidence event is an event where the signal is reflected back through an object, and the arrival time of the backscattering coincidence event is longer than the arrival time of a directly transmitted signaling event. Therefore, by setting an appropriate arrival time threshold, it is possible to distinguish the directly transmitted signaling events from the backscattering coincidence events. Correspondingly, there is a loss of energy during a reflection process, and the energy of the backscattering coincidence event is typically weaker than the energy of the directly transmitted signaling event. Therefore, the backscattering coincidence event is screened out by setting a suitable energy threshold. Similarly, the lutetium background event data is screened out by setting a suitable energy threshold.

The first preset energy window refers to an energy interval for determining whether the single event data that occurs later is the transmission data. The second preset energy window refers to an energy interval for determining whether the single event data that occurs first is the transmission data. The first preset energy window and the second preset energy window may be set based on experience or demands.

The first preset time window refers to a preset time interval for distinguishing whether the single event data is the transmission data. The first preset time window may be set based on experience or demands.

In some embodiments, the processing device 120 presets parameters in the first preset rule (the first preset energy window, the second preset energy window, the first preset time window, etc.), compares the single event data with the first preset rule, and if the single event data coincides with the first preset rule, it is determined to be the transmission data. Different transmission data (e.g., the backscattering coincidence event data, the lutetium background event data, etc.) may be screened by setting different parameters in the first preset rule (the first preset energy window, the second preset energy window, the first preset time window, etc.).

For example, the processing device 120 sets the first preset energy window to 250 keV-380 keV, the second preset energy window to 140 keV-250 keV, and the first preset time window to a theoretical arrival time+30. The theoretical arrival time refers to an arrival time of a single event to the detector, which is obtained based on a ratio of a flying distance of the single event (a distance between an annihilation position of the single event and a detection position) to the light speed, o indicates a system Gaussian time distribution standard deviation, σ=FWHM/2.355, and FWHM indicates a half-height full-width of a Gaussian function. Two different single events are selected from the single event data, and a single event 2 occurs before a single event 1. The energy of the single event 1 is compared with the first preset energy window, the energy of the single event 2 is compared with the second preset energy window, a difference between the arrival times of the single event 1 and the single event 2 are compared with the first preset time window, and if the energy of the single event 1 is within the first preset energy window, the energy of the single event 2 is within the second preset energy window, and the difference between the arrival times of the single event 1 and the single event 2 is within the first preset time window, then it is determined that the data corresponding to the single event 1 and the single event 2 belongs to the backscattering coincidence event data in the transmission data. Backscattering coincidence event data determined in this way may be also referred to as double backscattering coincidence event data determined using a dual-coincidence strategy. As used herein, the double backscattering coincidence event data relates to backscattering coincidence events defined by two single events (i.e., backscattering coincidence events detected by the dual-coincidence strategy).

For another example, the processing device 120 sets the first preset energy window to be 0 keV-1000 keV, the second preset energy window to be 100 keV-350 keV, and the first preset time window to be the theoretical arrival time±3σ. Two different single events are selected from the single event data, a single event 4 occurs before a single event 3. The energy of single event 3 is compared with the first preset energy window, the energy of single event 4 is compared with the second preset energy window, and a difference between the arrival time of the single event 3 and the arrival time of the single event 4 is compared with the first preset time window. If the energy of the single event 3 is within the first preset energy window, the energy of the single event 4 is within the second preset energy window, and the difference between the single event 3 and the single event 4 is within the first preset energy window, then it is determined that the data corresponding to the single event 3 and the single event 4 belongs to the lutetium background event data in the transmission data.

In some embodiments, the processing device 120 simultaneously collects the single event data and applies the single event data to the determinations of the backscattering coincidence event data and the lutetium background event data in the transmission data. Alternatively, the processing device 120 separately collects the single event data and applies them to the determination of the backscattering coincidence event data and the lutetium background event data in the transmission data, respectively.

In some embodiments of the present disclosure, by obtaining the single event data of the target object, and determining the single event data that coincides with the first preset rule as the transmission data, the single event data is comprehensively screened from the dimensions of the energy and the arrival time, and the noise and the interference signals are excluded, which improves the accuracy and the reliability of the projection data screening.

In 330, the attenuation-corrected radiological image may be obtained by performing the attenuation correction and reconstruction based on the transmission data and the radiological coincidence event data. In some embodiments, operation 330 is performed by the reconstruction module 230.

The radiological image refers to an image obtained by medical radiology techniques for diagnosing and evaluating diseases. For example, a PET image, etc.

The attenuation correction refers to a correction of errors in a medical image due to photon attenuation. Understandably, in an imaging process of the radiological image, an interaction between the emitted photons and the surrounding tissues can generate the photons. These photons are subjected to different degrees of attenuation as they pass through the body tissues, and these attenuations need to be corrected to obtain more accurate radiological images. The attenuation correction may remove the attenuation effect of the photons from different tissues, so as to obtain more accurate radiological images.

In some embodiments, the processing device 120 obtains the attenuation-corrected radiological image by performing the attenuation correction and reconstruction based on the transmission data and the radiological coincidence event data through a preset manner for attenuation correction. The preset manner for attenuation correction may be set based on experience or needs.

In some embodiments, the processing device 120 uses a first machine learning model to obtain the attenuation-corrected radiological image based on the transmission data and the radiological coincidence event data. In some embodiments, the first machine learning model includes, but is not limited to, a convolutional neural network (CNN) model, a recurrent neural network (RNN), a generative adversarial network (GAN) model, a long short-term memory network (LSTM) model, a transformer model, etc. Specifically, an input to the first machine learning model is the transmission data and the radiological coincidence event data, and an output of the first machine learning model is the attenuation-corrected radiological image. The first machine learning model may reconstruct the attenuation-corrected radiological image based on the transmission data and the radiological coincidence event data.

In some embodiments, the first machine learning model is obtained by training an initial first machine learning model using a plurality of first training samples with first labels. Specifically, the first training samples with the first labels are input to the initial first machine learning model, and parameters of the initial first machine learning model are updated through training to obtain the trained first machine learning model. The first training samples of the first machine learning model include sample transmission data and sample radiological coincidence data, and the first training labels include sample radiological images. The sample transmission data and the sample radiological coincidence event data may be historical data collected by a PET imaging device. The sample radiology images may be gold standard radiology images after attenuation correction based on the sample transmission data and the sample radiology coincidence event data.

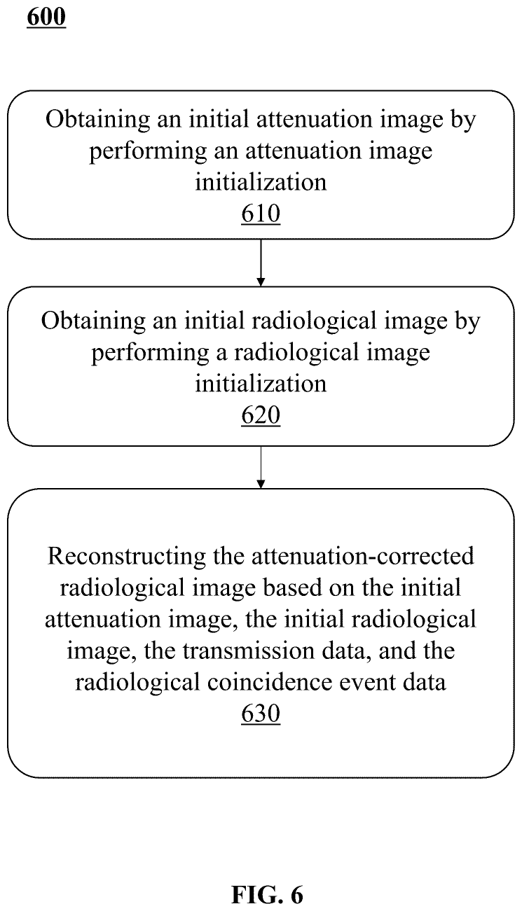

In some embodiments, the processing device 120 performs an iterative reconstruction on the radiological image based on the attenuation image to obtain the attenuation-corrected radiological image. More descriptions of the performing the iterative reconstruction on the radiological image based on the attenuation image to obtain the attenuation-corrected radiological image may be found in FIG. 6 and the related descriptions.

FIG. 6 is a flowchart illustrating an exemplary process for obtaining an attenuation-corrected radiological image of a target object according to some embodiments of the present disclosure. In some embodiments, operation 330 may be performed according to process 600 in FIG. 6. As shown in FIG. 6, an iterative reconstruction process 600 may include one or more of the following operations:

In 610, an initial attenuation image may be obtained by performing an attenuation image initialization.

The attenuation image refers to an image that reflects an attenuation feature of the target object. The initial attenuation image refers to an initially obtained attenuation image.

In some embodiments, the processing device 120 assigns an average value to each pixel value in a preset image (e.g., a mask image), performs the attenuation image initialization, and obtains the initial attenuation image. The average value may be preset based on experience or demands. For example, the processing device 120 assigns an attenuation factor of water to each pixel value in the preset image, performs the attenuation image initialization, and obtains the initial attenuation image.

In some embodiments, the processing device 120 determines a boundary of the target object in the preset image based on the transmission data, and then assigns the average value to each pixel value within the boundary. For example, the processing device 120 determines a plurality of data points associated with the boundary based on the transmission data, and then assigns the attenuation factor of water to each pixel value within the boundary.

In 620, an initial radiological image may be obtained by performing a radiological image initialization.

The initial radiological image refers to an initially obtained radiological image. More descriptions about the radiological image may be found in the preceding related

In some embodiments, the processing device 120 assigns the average value to each pixel value in the preset image for the radiological image initialization to obtain the initial radiological image. The average value may be preset based on experience or demands. In some embodiments, the processing device 120 determines the boundary of the target object in the preset image based on radiological coincidence event data, and then assigns the average value to each pixel value within the boundary. More descriptions can be found in the foregoing descriptions with respect to the attenuation image initialization.

In 630, the attenuation-corrected radiological image may be reconstructed based on the initial attenuation image, the initial radiological image, the transmission data, and the radiological coincidence event data.

In some embodiments, the processing device 120 performs the iterative reconstruction using a preset algorithm based on the initial attenuation image, the initial radiological image, the transmission data, and the radiological coincidence event data to obtain the attenuation-corrected radiological image. The preset algorithms may be preset based on experience or demands, for example, the preset algorithms may include a Monte Carlo manner, etc.

The iterative reconstruction refers to a process of multiple rounds of iteration, in which at least a portion of an output of each round is used as a portion of an input of the next round.

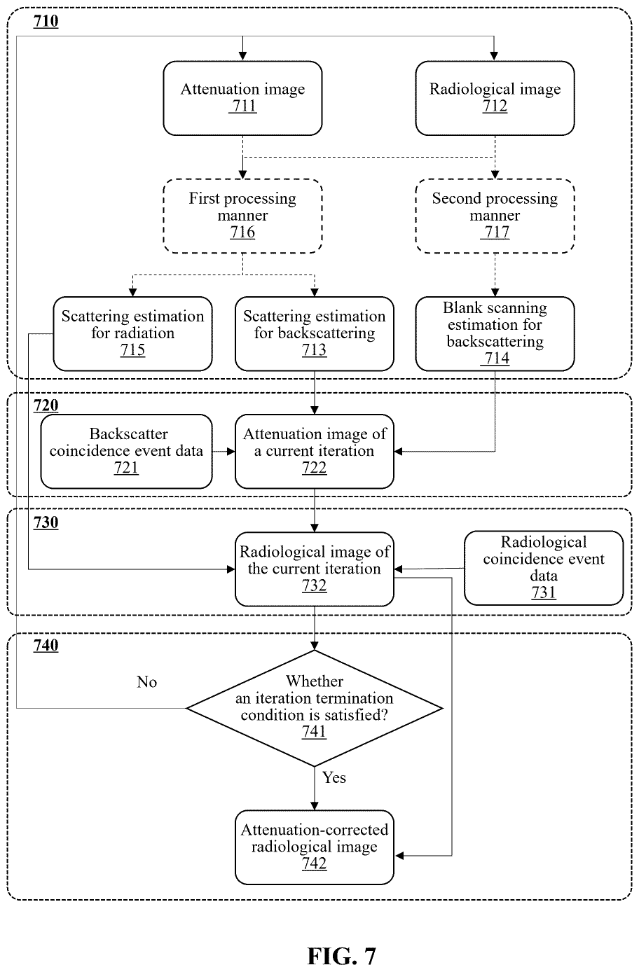

For example, in a process of iteratively reconstructing the attenuation-corrected radiological images, first an initial attenuation image and an initial radiological image are obtained based on an initialization of attenuation images and an initialization of radiological images as iterative inputs for the first round of iteration; in each round of iteration, the iterative input is analyzed and reconstructed to obtain an iterative attenuation image and an iterative radiological image, which are used to update the iterative input of the next iteration. The specific operation of iteration may be found in related contents in FIGS. 7-9.

In some embodiments, the processing device 120 uses a second machine learning model to obtain the attenuation-corrected radiological image based on the initial attenuation image, the initial radiological image, the transmission data, and the radiological coincidence event data. In some embodiments, the second machine learning model includes, but is not limited to, a CNN model, an RNN, a GAN model, an LSTM model, a transformer model, etc. Specifically, the input of the second machine learning model is the initial attenuation image, the initial radiological image, the transmission data, the radiological coincidence event data, and the output of the second machine learning model is the attenuation-corrected radiological image. The second machine learning model may iteratively reconstruct the initial attenuation image and the initial radiological image based on the transmission data and the radiography coincidence event data, and obtain the attenuation-corrected radiological image.

In some embodiments, the second machine learning model is obtained by training an initial second machine learning model using a plurality of second training samples with second labels. Specifically, the second training samples with the second labels are input to the initial second machine learning model, and parameters of the initial second machine learning model are updated through training to obtain the trained second machine learning model. The second training samples of the second machine learning model include sample initial attenuation images, sample initial radiological images, sample transmission data, and sample radiological coincidence event data, and the second training labels include sample radiological images. The sample initial attenuation images and the sample initial radiological images are obtained based on the sample transmission data, the sample radiological coincidence event data. More descriptions of obtaining the sample initial attenuation images and the sample initial radiological images may be found in the relevant descriptions of operation 610 and operation 620. More descriptions of the sample transmission data, the sample radiological coincidence event data, and the sample radiological image may be found in the relevant descriptions of operation 330.

In some embodiments of the present disclosure, by obtaining the transmission data and the radiological coincidence event data and performing the attenuation correction and reconstruction, the attenuation-corrected radiological image is obtained. In this way, the accurate attenuation-corrected radiological image is obtained without the aid of a CT image, thereby avoiding an impact of CT image artifacts and other errors on a correction result, and the process does not increase a complexity of the system, does not increase a scanning time, and does not increase a radiation dose to a patient, while considering a system design and a convenience of user operation, and avoiding an impact on a health of the patient.

FIG. 7 is a schematic diagram illustrating an exemplary process for obtaining an attenuation-corrected radiological image by performing an iterative reconstruction based on backscattering coincidence event data of the target object according to some embodiments of the present disclosure. In some embodiments, operation 630 may be performed according to FIG. 7.

In 710, a scattering estimation for backscattering 713, a blank scanning estimation for backscattering 714, and a scattering estimation for radiation 715 of a target object may be obtained based on an attenuation image, and a radiological image.

The attenuation image of a first iteration may be an initial attenuation image, and the radiological image of the first iteration may be an initial radiological image. The attenuation image of the iterations other than the first iteration may be an attenuation image 711 of the previous iteration, and the radiological image of the iterations other than the first iteration may be a radiological image 712 of the previous iteration.

The scattering estimation for backscattering 713 refers to a distribution estimation of scattering events present in the backscattering coincidence event data. The scattering estimation for radiation 715 refers to a distribution estimation of coincidence events present in the scanning data of the target object. The scanning data refers to data obtained by a scanning device in real time that is used to reconstruct the attenuation-corrected radiological image. For example, a count of all events obtained by scanning the target object, etc. The scattering estimation for backscattering 713 and the scattering estimation for radiation 715 may be expressed in the form of sinograms.

In some embodiments, the processing device 120 obtains the scattering estimation for backscattering 713 for and/or the scattering estimation for estimation 715 based on the attenuation image 711 of the previous iteration, the radiological image 712 of the previous iteration, through a preset algorithm. The preset algorithm may be preset based on experience or demands, for example, the preset algorithm may include a Monte Carlo manner, etc.

In some embodiments, the processing device 120 obtains the scattering estimation for backscattering 713 and/or the scattering estimation for radiation 715 based on the attenuation image 711 of the previous iteration and the radiological image 712 of the previous iteration by a first processing manner 716. The first processing manner 716 refers to a processing manner for obtaining the scattering estimation.

In some embodiments, the first processing manner 716 includes one or more of the Monte Carlo manner, a conventional single scatter simulation (SSS) manner, an Energy-based scatter estimation (EBS) manner, etc.

In some embodiments, the first processing manner 716 includes processing the attenuation image of the previous iteration and the radiological image of the previous iteration using a third machine learning model to obtain the scattering estimation for backscattering and the scattering estimation for radiation.

In some embodiments, the third machine learning model includes, but is not limited to, a CNN model, an RNN model, a GAN model, an LSTM model, a transformer model, etc. Specifically, an input to the third machine learning model may be the attenuation image of the previous iteration and the radiological image of the previous iteration, and an output of the third machine learning model may be the scattering estimation for backscattering, and the scattering estimation for radiation. The third machine learning model may analyze the attenuation image of the previous iteration and the radiological image of the previous iteration, and obtain the scattering estimation for backscattering and the scattering estimation for radiation for the current iteration.

In some embodiments, the third machine learning model is obtained by training an initial third machine learning model using a plurality of third training samples with third labels. Specifically, the third training samples with the third labels are input to the initial third machine learning model, and parameters of the initial third machine learning model are updated by training to obtain the trained third machine learning model. The third training samples of the third machine learning model include sample attenuation images, sample radiological images, and the third training labels include sample scattering estimation for backscattering, and sample scattering estimation for radiation. Sample transmission data and sample radiological coincidence event data may be historical data collected by a PET imaging device. The sample scattering estimation for backscattering and the sample scattering estimation for radiation may be determined by analyzing the sample attenuation image and the sample radiological image by other first processing manners.

The blank scanning estimation for backscattering 714 refers to a statistical distribution of backscattering coincidence events simulated under a blank scan condition—i.e., without any attenuating or scattering material. The simulation takes the annihilation photon emission distribution of the target object as the source, and computes the detector-induced backscatter response without performing an actual scan of the object. The blank scanning estimation for backscattering 714 may be expressed in a form of a sinogram. The blank scanning estimation for backscattering 714 may be expressed in a form of a sinogram.

In some embodiments, the processing device 120 obtains the blank scanning estimation for backscattering 714 based on the attenuation image 711 of the previous iteration, the radiological image 712 of the previous iteration, by processing using a second processing manner 717.

The second processing manner 717 refers to a processing manner for obtaining a blank scanning estimation.

In some embodiments, the second processing manner 717 includes at least one of a Monte Carlo manner, a look-up table manner.

The look-up table refers to a data table reflecting a probability distribution of an occurrence of backscattering on a LOR. In some embodiments, the look-up table includes a probability distribution of the backscattering of events on each LOR of the PET system being detected by remaining LORs of the PET system. More contents on the LORs may be found in FIG. 3 and the related descriptions.

The look-up table may be represented in a matrix form. For example, if there are M LORs, each of which corresponds to an M*1 sinogram. The sinogram reflects the probability distribution of the backscattering of events on each LOR of the PET system being detected by remaining LORs of the PET system, and the look-up table is an M*M matrix, including M count of M*1 sinograms. M is an integer.

Specifically, in some embodiments, the processing device 120 obtains the look-up table. In some embodiments, the processing device 120 obtains the look-up table through a physical model, the Monte Carlo manner, or other mathematical modeling techniques. For example, the processing device 120 simulates all probabilities of a radiological event incident along a direction of a certain LOR being detected by other LORs by means of the physical model (parameters of the model need to be set autonomously according to an actual scenario), the Monte Carlo manner, etc., and taking the probabilities as a column of data to be filled in the look-up table, traverses all the LORs and constructs the look-up table.

Further, in some embodiments, the processing device 120 simplifies the look-up table based on a symmetry of a positron emission computed tomography system and/or a merging of the LORs.

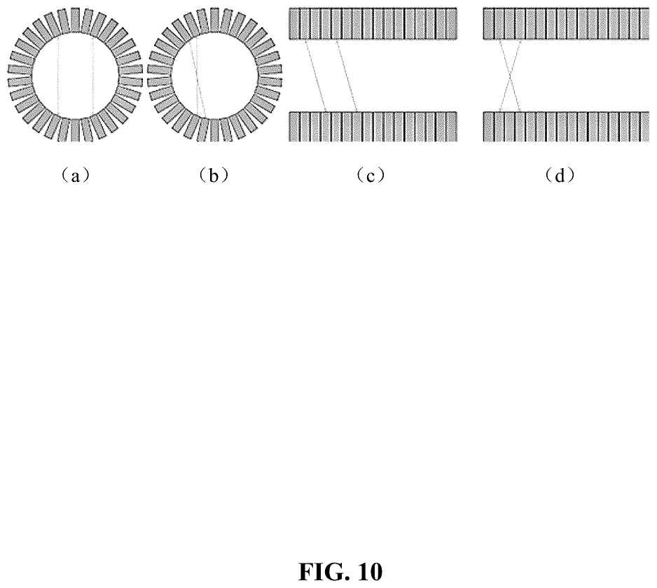

It may be appreciated that the positron emission computed tomography system possesses the symmetry, and the symmetry includes a cross-sectional reflection symmetry of the detector (as shown in (a) in FIG. 10), a cross-sectional rotation symmetry (as shown in (b) in FIG. 10), an axial parallel symmetry (as shown in (c) in FIG. 10), and an axial reflection symmetry (as shown in (d) in FIG. 10). In some embodiments, the processing device 120 reserves, based on the symmetry of the positron emission computed tomography system, among the plurality of LORs where a symmetrical relationship exists, only data of any one LOR (i.e., the data of probability distribution of the backscattering of events on each LOR of the PET system being detected by remaining LORs) in the look-up table, and deletes the data of the other LORs, thereby realizing a simplification of the look-up table.

In some embodiments, the processing device 120 merges the plurality of LORs into a single LOR based on the merging of LORs, retaining only the data of that LOR in a look-up table, realizing a simplification of the look-up table. The merging of the LORs may be realized by a merging of detection units of a corresponding detector. For example, the merging of the LORs is realized by merging 4 adjacent detector units, i.e., merging the LORs detected by 4 adjacent detector units into 1. Specific merging rules may be preset based on experience or demands.

Furthermore, in some embodiments, the processing device 120 determines the blank scanning estimation for backscattering 714 based on the simplified look-up table, and the scanning data.

In some embodiments, the processing device 120 determines the blank scanning estimation for backscattering 714 based on the simplified look-up table and the scanning data by calculation. For example, the processing device 120 calculates a sum of an event count on each LOR in the scanning data, and a product of the probability distribution of the corresponding LOR over the other LORs in the simplified look-up table (i.e., the sinogram of the aforementioned LORs), and determines the sum as the blank scanning estimation for backscattering.

In some embodiments of the present disclosure, by constructing and simplifying the look-up table and determining the blank scanning estimation for backscattering 714, the probability distribution of the backscattering of events on the LOR being detected by remaining LORs is obtained by simulation, and thus obtaining an accurate blank scanning estimation for backscattering.

In some embodiments, the second processing manner 717 includes processing the attenuation image of the previous iteration and the radiological image of the previous iteration using a fourth machine learning model, so as to obtain a blank scanning estimation for backscattering.

In some embodiments, the fourth machine learning model includes, but is not limited to, the CNN model, the RNN model, the GAN model, the LSTM model, the transformer model, etc. Specifically, an input to the fourth machine learning model is the attenuation image of the previous iteration and the radiological image of the previous iteration, and an output of the fourth machine learning model is the blank scanning estimation for backscattering. The fourth machine learning model may analyze the attenuation image of the previous iteration and the radiological image of the previous iteration, and obtains the blank scanning estimation for backscattering for a current round of iteration.

In some embodiments, the fourth machine learning model is obtained by training an initial fourth machine learning model using a plurality of fourth training samples with fourth labels. Specifically, the fourth training samples with the fourth labels are input to an initial fourth machine learning model, and parameters of the initial fourth machine learning model are updated through training to obtain the trained fourth machine learning model. The fourth training samples of the fourth machine learning model include sample attenuation images, sample radiological images, and the fourth training labels include sample scattering estimation for backscattering, and sample scattering estimation for radiation. The sample transmission data, the sample radiological coincidence event data may be historical data obtained by the PET imaging device. The blank scanning estimation for backscattering may be determined by analyzing the sample attenuation image and the sample radiological image by other second processing manners.

In some embodiments of the present disclosure, obtaining the scattering estimation for backscattering 713, the scattering estimation for radiation 715, and the blank scanning estimation for backscattering 714 by processing the attenuation image 711 of the previous iteration, the radiological image 712 of the previous iteration through the first processing manner 716 and the second processing manner 717 makes a determination process of the scattering estimation and the blank scanning estimation efficient, accurate, and convenient, which facilitates subsequent updating and reconstruction of the corresponding images.

In 720, an attenuation image of a current iteration 722 may be obtained based on the scattering estimation for backscattering 713, the blank scanning estimation for backscattering 714, and the transmission data. The transmission data may include backscattering coincidence event data 721 of the target object.

In some embodiments, as shown in (a) in FIG. 11, when iterating the attenuation image based on the backscattering coincidence event data, the attenuation image is iteratively calculated according to Equation (1):

$${μ}_{bs}^{k + 1} = {μ}_{bs}^{k} + \frac{{H}^{T} [ {B}_{bs}^{k} {e}^{- H {μ}_{bs}^{k}} ( 1 - \frac{{y}_{bs}}{{B}_{bs}^{k} {e}^{- H {μ}_{bs}^{k}} + {s}_{bs}^{k} + {r}_{bs}} ) ]}{{H}^{T} [ \frac{{( {B}_{bs}^{k} {e}^{- H {μ}_{bs}^{k}} )}^{2}}{{B}_{bs}^{k} {e}^{- H {μ}_{bs}^{k}} + {s}_{bs}^{k} + {r}_{bs}} H \cdot 1 ]} , \qquad ( 1 )$$

where

$${μ}_{bs}^{k + 1}$$

denotes the attenuation image of the current iteration.

$${μ}_{bs}^{k}$$

denotes the attenuation image of the previous iteration,

$${B}_{bs}^{k}$$

denotes the plank scanning estimation for backscattering, H denotes a system matrix, y_{bs} denotes the backscattering coincidence events in the transmission data,

$${s}_{bs}^{k}$$

denotes the scattering estimation for backscattering, r_{bs} denotes a random event estimation for backscattering, the random event estimation for backscattering referring to a distribution estimation of random events present in the backscattering coincidence event data, which is obtained by a delay window method (DWM), a singles rate (SR), etc. k denotes a current iteration count.

In 730, a radiological image of the current iteration 732 may be obtained based on the attenuation image of the current iteration 722, the scattering estimation for radiation 715, and the radiological coincidence event data 731.

In some embodiments, the processing device 120 obtains the radiological image of the current iteration 732 based on the attenuation image of the current iteration 722, the scattering estimation for radiation 715, and the radiological coincidence event data 731. In some embodiments, the radiological image is calculated iteratively according to Equation (2):

$${λ}^{k + 1} = \frac{{λ}^{k}}{{( {A}^{k} H )}^{T} \cdot 1} {H}^{T} \frac{{y}_{emission}}{H {λ}^{k} + \frac{{ss}^{k} + rr}{{A}^{k} \cdot 1}} , {A}^{k} = diag ( {e}^{- H {μ}_{*}^{k}} ) , \qquad ( 2 )$$

where λ^{k+1} denotes the radiological image of the current iteration, λ^{k} denotes the radiological image of the previous iteration, yₑₘᵢₛₛᵢₒₙ denotes the radiological coincidence event data, ss denotes a scattering estimation of radiological events, the scattering estimation of radiological events refers to a distribution estimation of scattering events present in the radiological coincidence event data, and rr denotes the random event estimation for radiological events, which is obtained by the DWM, the SR, etc.

$${μ}_{*}^{k} is {μ}_{bs}^{k} or {μ}_{all}^{k} .$$

In 740, whether an iteration termination condition 741 is satisfied may be determined. In response to that the iteration termination condition 741 is not satisfied, the next iteration is performed. In response to that the iteration termination condition 741 is satisfied, the radiological image of the current iteration 732 is determined as an attenuation-corrected radiological image 742.

If an iteration situation satisfies a preset iteration condition, the iteration terminates, and the attenuation correction finishes. The preset iteration condition may be that the iteration converges, a preset count of iterations is reached, a difference between the radiological images in the two adjacent iterations is less than a certain threshold, a difference between the attenuation effect sinograms in the two adjacent iterations is less than a certain threshold, etc. The attenuation effect sinogram refers to a forward projection of the attenuation image.

The attenuation-corrected radiological image refers to a radiological image that completes the attenuation correction, which is the radiological image that is output in the last iteration.

In some embodiments of the present disclosure, by obtaining the scattering estimation for backscattering, the blank scanning estimation for backscattering, and the scattering estimation for radiation based on the attenuation image of the previous iteration and the radiological image of the previous iteration; and then obtaining the attenuation image and the radiological image of the current iteration, the iteration is terminated after the iteration termination condition(s) are satisfied, and the attenuation image and radiological image are updated and reconstructed based on real-time data, which makes the attenuation correction process more accurate and efficient.

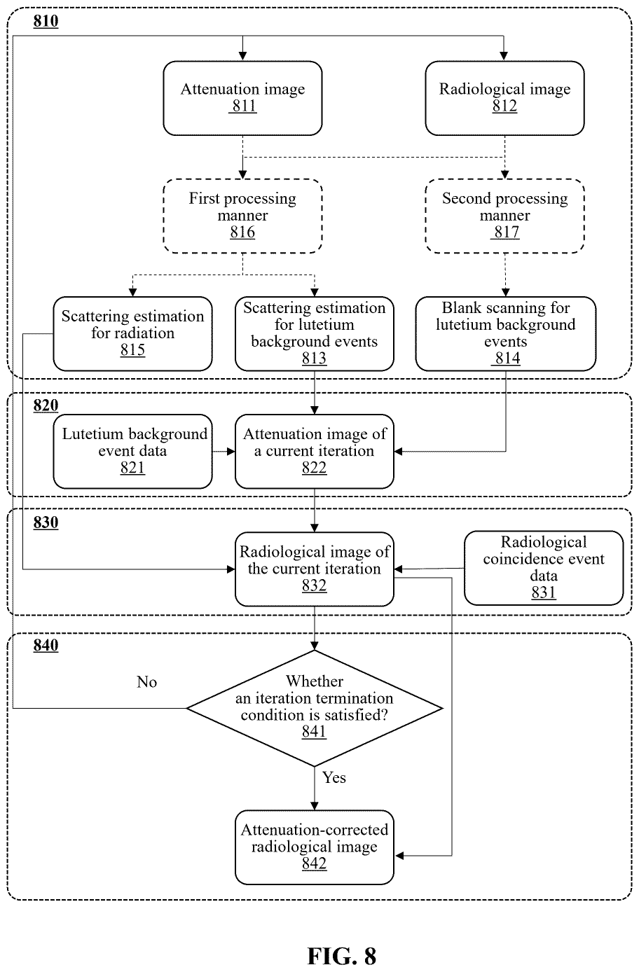

FIG. 8 is a schematic diagram illustrating an exemplary process for obtaining an attenuation-corrected radiological image by performing an iterative reconstruction based on lutetium background event data according to some embodiments of the present disclosure. In some embodiments, operation 630 may be performed according to operations in FIG. 8.

In 810, a scattering estimation for lutetium background events 813, a blank scanning for the lutetium background events 814, and a scattering estimation for radiation 815 of the target object may be obtained based on an attenuation image and a radiological image.

The attenuation image of a first iteration is an initial attenuation image, and the radiological image of the first iteration is an initial radiological image. The attenuation image of the iterations other than the first iteration is an attenuation image 811 of the previous iteration, and the radiological image of the iterations other than the first iteration is the radiological image 812 of the previous iteration.

The scattering estimation for lutetium background events 813 refers to an estimation of a distribution of scattering events present in lutetium background event data. The scattering estimation for radiation 815 refers to a distribution estimation of coincidence events present in scanning data of the target object. The scattering estimation for lutetium background events 813 and the scattering estimation for radiation 815 may be expressed in a form of a sinogram.

In some embodiments, the processing device 120 obtains, using a preset algorithm, the scattering estimation for the lutetium background events 813 and/or the blank scanning for the lutetium background events 814 based on the attenuation image 811 of the previous iteration and the radiological image 812 of the previous iteration. The preset algorithm is preset based on experience or demands, for example, the preset algorithm includes the Monte Carlo manner, etc.

In some embodiments, the processing device 120 obtains, using a first processing manner, the scattering estimation for the lutetium background events 813 and/or the scattering estimation for radiation 815 based on the attenuation image 811 of the previous iteration and the radiological image 812 of the previous iteration. More descriptions of the first processing manner may be found in the description associated with operation 710.

The blank scanning for the lutetium background events 814 refers to a distribution estimation of the lutetium background events when there are no target objects during a generation of lutetium spontaneous background radiation. In some embodiments, the processing device 120 obtains the blank scanning for the lutetium background events 814 based on the attenuation image 811 of the previous iteration, the radiological image 812 of the previous iteration, by processing using the second processing manner 817. More descriptions of the second processing manner may be found in the description associated with operation 710.

In 820, an attenuation image of a current iteration 822 may be obtained based on the scattering estimation of the lutetium background events 813, the blank scanning for the lutetium background events 814, and the transmission data. The transmission data includes lutetium background event data 821.

In some embodiments, as shown in (b) in FIG. 11, when iterating the attenuation image based on the lutetium background event data, the attenuation image is calculated iteratively according to Equation (3):

$${μ}_{Lu}^{k + 1} = {μ}_{Lu}^{k} + \frac{{H}^{T} [ {B}_{Lu} {e}^{- H {μ}_{Lu ( 1 - \frac{{y}_{Lu}}{{B}_{Lu} {e}^{- H {μ}_{Lu}^{k} + {s}_{Lu}^{k} + {r}_{Lu}}} )}^{k}} ]}{{H}^{T} [ \frac{{( {B}_{Lu} {e}^{- H {μ}_{Lu}^{k}} )}^{2}}{{B}_{Lu} {e}^{- H {μ}_{Lu + {s}_{Lu}^{k} + {r}_{Lu}}^{k}}} H \cdot 1 ]} , \qquad ( 3 )$$

where

$${μ}_{Lu}^{k + 1}$$

denotes the attenuation image of the current iteration,

$${μ}_{Lu}^{k}$$

denotes the attenuation image of the previous iteration, B_{Lu} denotes the blank scanning for the lutetium background events, H denotes the system matrix, y_{Lu} denotes the lutetium background events in the transmission data,

$${s}_{Lu}^{k}$$

denotes the scattering estimation for lutetium background events, the scattering estimation for lutetium background events referring to a distribution estimation of the scattered events present in the lutetium background event data, r_{Lu} denotes a random event estimation for the lutetium background events. The random event estimation refers to a distribution estimation of random events present in the lutetium background event data, which is obtained by the DWM, the SR, etc. k denotes the current iteration count. The blank scanning for the lutetium background events is achieved by a blank scanning of the scanning device 110. The blank scanning refers to a direct scanning on air without the target object such as a human body or a molded body, which is regarded as the air.

In 830, a radiological image of a current iteration 832 may be obtained based on the attenuation image of the current iteration 822, the scattering estimation for radiation 815 of the target object, and the radiological coincidence event data 831. More descriptions of obtaining the radiological image of the current iteration based on the attenuation image of the current iteration, the scattering estimation for radiation, and the radiological coincidence event data may be found in the descriptions related to operation 730, which is not repeated herein.

In 840, whether an iteration termination condition is satisfied may be determined. In response to that the iteration termination condition is not satisfied, the process may proceed to a next iteration; or in response to that the iteration termination condition is satisfied, the radiological image of the current iteration 832 may be designated as the attenuation-corrected radiological image. More descriptions of determining whether the iteration termination condition is satisfied may be found in the relevant descriptions of operation 740, which is not repeated here.

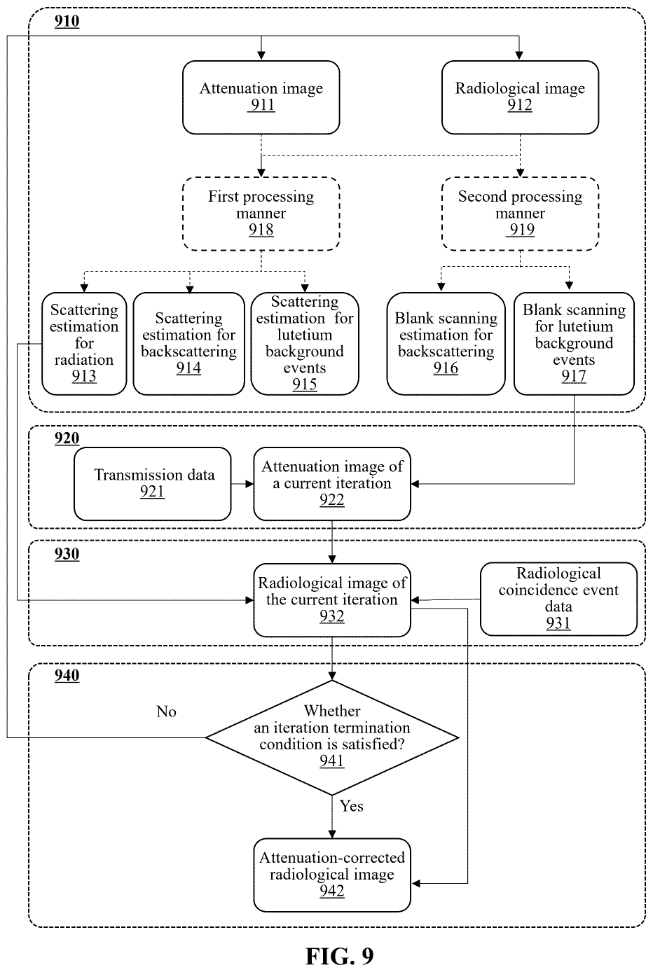

FIG. 9 is a schematic diagram illustrating an exemplary process for obtaining an attenuation-corrected radiological image by performing an iterative reconstruction based on lutetium background event data and backscattering coincidence event data according to some embodiments of the present disclosure. In some embodiments, operation 630 may be performed according to FIG. 9.

In 910, a scattering estimation for backscattering 914, a blank scanning estimation for backscattering 916, a scattering estimation for lutetium background events 915, a blank scanning for lutetium background events 917, and a scattering estimation for radiation 913 of a target object may be obtained based on an attenuation image and a radiological image.

The attenuation image of a first iteration is an initial attenuation image, and the radiological image of the first iteration is an initial radiological image. An attenuation image of the iterations other than the first iteration is an attenuation image 911 of the previous iteration, and a radiological image of the iterations other than the first iteration is a radiological image 912 of the previous iteration.

More descriptions of the scattering estimation for backscattering 914, the blank scanning estimation for backscattering 916, and the scattering estimation for radiation 913 may be found in the related descriptions of operation 710.

More descriptions of the scattering estimation for lutetium background events 915 and the blank scanning for lutetium background events 917 may be found in related descriptions of operation 810.

In 920, an attenuation image of a current iteration 922 may be obtained based on the scattering estimation for backscattering 914, the blank scanning estimation for backscattering 916, the scattering estimation for the lutetium background events 915, the blank scanning for the lutetium background events 917, and the transmission data 921.

In some embodiments, as shown in (c) in FIG. 11, when an attenuation image iteration is performed based on the backscattering coincidence event data and the lutetium background event data together in the transmission data, the attenuation image is calculated iteratively according to Equation (4):

$${μ}_{all}^{k + 1} = {μ}_{all}^{k} + \frac{{H}^{T} [ {e}^{- H {μ}_{all ( 1 - \frac{{y}_{all}}{{B}_{all}^{k} {e}^{- H {μ}_{all + {s}_{all}^{k} + {r}_{all}}^{k}}} )}^{k}} ]}{{H}^{T} [ \frac{{( {B}_{all}^{k} {e}^{- H {μ}_{all}^{k}} )}^{2}}{{B}_{all}^{k} {e}^{- H {μ}_{all + {s}_{all}^{k} + {r}_{all}}^{k}}} H \cdot 1 ]} , \qquad ( 4 )$$

where

$${μ}_{all}^{k + 1}$$

denotes the attenuation image of the current iteration,

$${μ}_{all}^{k}$$

denotes the attenuation image of the previous iteration,

$${B}_{all}^{k} = {B}_{bs}^{k} + {B}_{Lu} , {y}_{all} = {y}_{bs} + {y}_{Lu} , {s}_{all}^{k} = {s}_{bs}^{k} + {s}_{Lu}^{k} , {r}_{all} = {r}_{bs} + {r}_{Lu} .$$

In 930, a radiological image of the current iteration 932 may be obtained based on the attenuation image of the current iteration 922, the scattering estimation for radiation 913 of the target object, and the radiological coincidence event data 931. More descriptions of obtaining the radiological image of the current iteration based on the attenuation image of the current iteration, the scattering estimation for radiation of the target object, and the radiological coincidence event data may be found in the relevant description of operation 730, which is not repeated here.

In 940, whether an iteration termination condition is satisfied may be determined. In response to that the iteration termination condition is not satisfied, the process may proceed to a next iteration; or in response to that the iteration termination condition is satisfied, the radiological image of the current iteration 932 may be designated as an attenuation-corrected radiological image 942. More descriptions of determining whether the iteration termination condition is satisfied may found in the relevant descriptions of operation 740, which is not repeated here.

In certain scenarios, low tracer activity in a scanning object, which results from an insufficient tracer dosage, low metabolic activity, or poor tracer uptake, can lead to a reduced number of backscattering coincidence events. This scarcity of valid data consequently compromises the statistical accuracy of the attenuation correction process and negatively impacts the quality of a reconstructed attenuation-corrected image. To address the above challenges, the present disclosure provides a ECT scanner introducing an external photon emission device. Some of the photons emitted from the photon emission device strike a detector of the ECT scanner and undergo Compton scattering. The scattered photons then traverse a scanning object, resulting in backscattering coincidence events. These backscattering coincidence events originating from the photon emission device, together with those originating from positive and negative electron annihilation coincidence events within the scanning object (e.g., the backscattering coincidence events described in FIG. 3), can be collectively utilized for performing attenuation correction on scan data of the scanning object.

FIG. 12 is a schematic diagram illustrating an exemplary ECT scanner 1200 according to some embodiments of the present disclosure. In some embodiments, the ECT scanner 1200 may be an exemplary embodiment of the scanning device 110 as described in connection with FIG. 1.

As shown in FIG. 12, the ECT scanner may include a detector 1210 and a photon emission device 1220. The detector 1210 defines an imaging field. The photon emission device 1220 is disposed outside the imaging field and configured to emit photons before and during an ECT scan. At least a portion of the photons enter the imaging field and strike the detector 1210.

In some embodiments, an energy of the photons is in a range from 430 keV to 650 keV. For example, the energy of the photons is 511 keV.

The photon emission device 1220 may be disposed anywhere outside the imaging field, as long as at least a portion of the photons emitted by the photon emission device 1220 can enter the imaging field and strike the detector 1210. For example, as shown in FIG. 12, the photon emission device 1220 is arranged at a central axis (e.g., the central axis OX of the detector 1210 shown in FIG. 12) of the imaging field and spaced apart from the detector 1210 along the central axis.

The ECT scanner 1200 may include any count of photon emission devices 1220. At least a portion of the photons emitted by each photon emission device 1220 enter the imaging field and strike the detector 1210.

In some embodiments, the photon emission device 1220 may include a photon emission source and one or more photon shielding structures. The photon emission source is configured to emit photons.

The photon shielding structure(s) are configured to shield photons emitted by the photon emission source along undesired directions, to prevent or reduce the dispersal of photons toward the scanning object and the surrounding environment. The photon shielding structure may be made from materials (e.g., lead, tungsten, or alloys thereof) with photon shielding capacity (e.g., with high density and high atomic number).

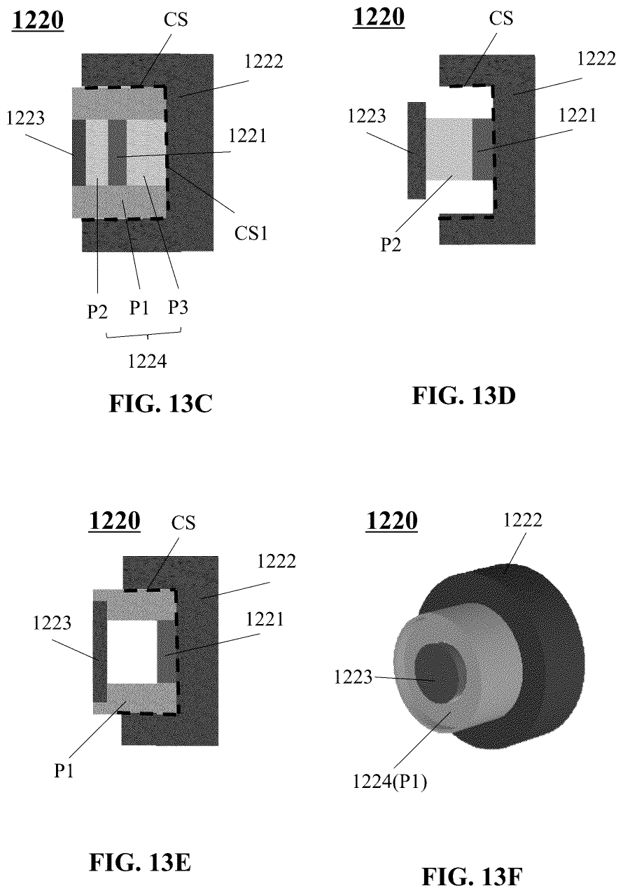

In some embodiments, the photon shielding structure(s) may include a first photon shielding structure and a second photon shielding structure, separated from each other. FIGS. 13A-13E are cross-section views illustrating exemplary photon emission devices 1220 according to some embodiments of the present disclosure. FIG. 13F is a schematic diagram illustrating a perspective view of an exemplary photon emission device 1220 according to some embodiments of the present disclosure. As shown in FIGS. 13A-13F, the photon emission device 1220 may include a photon emission source 1221 (not shown in FIG. 13F), a first photon shielding structure 1222, a second photon shielding structure 1223, and a fixing structure 1224.

In some embodiments, the photon emission source 1221 includes a point source, a linear source, a cylindrical source, or an array source. For example, the photon emission source 1221 is an array source including a plurality of point sources arranged in an array. In some embodiments, the photon emission source 1221 includes a housing that supports and/or accommodates the point source, the linear source, the array source, etc. The housing may be configured in any suitable shape, such as cylindrical, spherical, or cubic.

In some embodiments, the photon emission source 1221 may include at least one of a positron-emitting radionuclide or a single-photon-emitting radionuclide. The positron-emitting radionuclide (e.g., Na-22, Ge-68, etc.) emits a pair of 511 keV photons. The single-photon-emitting radionuclide (e.g., Co-57) emits a single photon. Some of the photons emitted by the positron-emitting radionuclide and/or the single-photon-emitting radionuclide may be emitted out from the photon emission device 1220 and move toward the detector 1210. For example, one photon of the pair of photons emitted by the positron-emitting radionuclide may move in a direction toward the detector 1210, while the other photon of the pair of photons is shieled by the photon shielding structure(s).

The first photon shielding structure 1222 may be disposed on a first side of the photon emission source 1221 facing away from the detector 1220. The first photon shielding structure 1222 is configured to shield photons emitted by photon emission source 1221 that move in the direction away from the detector 1220.

The second photon shielding structure 1223 may be disposed on a second side of the photon emission source 1221 facing the detector 1220. The second photon shielding structure 1223 is configured to shield photons emitted by photon emission source 1221 that move toward a scanning object 1230.

The fixing structure 1224 may secure positions of the photon emission source 1221 and the second photon shielding structure 1223 relative to the first photon shielding structure 1222. The fixing structure 1224 is made of a material (e.g., hard plastic, glass, foam plastic, etc.) that allows the photons to pass through.

In some embodiments, as shown in FIGS. 13A-13E, the first photon shielding structure 1222 includes a concave structure CS, and the photon emission source 1221 is secured within the concave structure CS. The concave structure CS is arranged on a side of the first photon shielding structure 1222 facing the detector 1210. The concave structure CS includes a bottom surface CS1 facing the detector 1210.

In some embodiments, as shown in FIGS. 13A and 13B, the photon emission source 1221 is secured to the bottom surface CS1 of the concave structure CS. The photon emission source 1221 and the bottom surface CS1 of the concave structure CS are connected and secured by means such as an adhesive bonding. In this way, the photon emission device 1220 features a structurally simple design that is readily manufacturable.

In some embodiments, as shown in FIG. 13C, the photon emission source 1221 is secured to be suspended in the concave structure CS. That is, the photon emission source 1221 doesn't contact with the bottom surface CS1 of the concave structure CS. In this way, photons are more likely to be emitted out from the photon emission device 1220.

In some embodiments, the second photon shielding structure 1223 is secured outside or within the concave structure and does not contact the bottom surface of the concave structure, and the second photon shielding structure 1223 is spaced apart from the photon emission source 1221. For example, as shown in FIG. 13A, the second photon shielding structure 1223 is secured outside the concave structure. As another example, as shown in FIG. 13B, the second photon shielding structure 1223 is secured within the concave structure CS and does not contact the bottom surface CS1 of the concave structure CS.

In some embodiments, the fixing structure 1224 comprises at least one of a first component or a second component. The first component is connected between the photon emission source 1221 and the first photon shielding structure 1222, and configured to secure the position of the photon emission source 1221 relative to the first photon shielding structure 1222 to avoid displacement of the photon emission source 1221 relative to the first photon shielding structure 1222. The first component is further connected between the second photon shielding structure 1223 and the first photon shielding structure 1222, and configured to secure the position of the second photon shielding structure 1223 relative to the first photon shielding structure 1222 to avoid displacement of the second photon shielding structure 1223 relative to the first photon shielding structure 1222. The second component is connected between the photon emission source 1221 and the second photon shielding structure 1223, and configured to secure the position of the second photon shielding structure 1223 relative to the photon emission source 1221 to avoid displacement of the second photon shielding structure 1223 relative to the photon emission source 1221.

In some embodiments, as shown in FIGS. 13A-13C, the fixing structure 1224 comprises a first component P1 and a second component P2. The first component P1 surrounds the photon emission source 1221. The first component P1 is connected between the photon emission source 1221 and the first photon shielding structure 1222. In some embodiments, the first component P1 may have a hollow structure. The inner surface of the first component P1 is connected to the photon emission source 1221, and the outer surface of the first component P1 is connected to the concave structure CS. The first component P1 is also connected between the second photon shielding structure 1223 and the first photon shielding structure 1222. The inner surface of the first component P1 is connected to at least a portion of surfaces of the second photon shielding structure 1223 other than a surface of the second photon shielding structure 1223 facing the detector 1210. In some embodiments, the first component P1 surrounds the second photon shielding structure 1223.

The second component P2 is connected between the photon emission source 1221 and the second photon shielding structure 1223. The second component P2 is connected to a side of the second photon shielding structure 1223 facing the photon emission source 1221. As shown in FIGS. 13A and 13B, when the photon emission source 1221 is secured to the bottom surface CS1 of the concave structure CS, a side of the photon emission source 1221 is secured to the bottom surface CS1 and the other side of the photon emission source 1221 away from the bottom surface CS1 is connected to the second component P2.

As shown in FIG. 13C, when the photon emission source 1221 is secured to be suspended in the concave structure CS, the fixing structure 1224 further comprises a third component P3. Specifically, the third component P3 is connected between the photon emission source 1221 and the bottom surface CS1, such that the photon emission source 1221 is spaced apart from the bottom surface CS1.

In some embodiments, as shown in FIG. 13D, the fixing structure 1224 only comprises the second component P2. A side of the photon emission source 1221 is secured to the bottom surface CS1 and the other side of the photon emission source 1221 away from the bottom surface CS1 is connected to a side of the second component P2 facing the bottom surface CS1. The other side of the second component P2 away from the bottom surface CS1 is further connected to the second photon shielding structure 1223 to secure the position of the second photon shielding structure 1223 relative to the first photon shielding structure 1222. Through this configuration, photons can be emitted to outside of the photon emission device 1220 through the air without obstruction.

In some embodiments, as shown in FIG. 13E, the fixing structure 1224 only comprises the first component P1. The photon emission source 1221 is secured to the bottom surface CS1. The first component P1 surrounds the photon emission source 1221. The first component P1 is connected between the photon emission source 1221 and the first photon shielding structure 1222. In some embodiments, the first component P1 may have a hollow structure. The inner surface of the first component P1 is connected to the photon emission source 1221, and the outer surface of the first component P1 is connected to the concave structure CS. The first component P1 is also connected between the second photon shielding structure 1223 and the first photon shielding structure 1222. The inner surface of the first component P1 is connected to at least a portion of surfaces of the second photon shielding structure 1223 other than a surface of the second photon shielding structure 1223 facing the detector 1210. In some embodiments, the first component P1 surrounds the second photon shielding structure 1223.

In some embodiments, the first component P1 and the second component P2 have different hardnesses. For example, as shown in FIG. 13A and FIG. 13B, the first component P1 is primarily configured to secure and support the photon emission source 1221, while the second component P2 is mainly employed to assist in positioning the second photon shielding structure 1223. Therefore, the hardness of the first component P1 may be greater than that of the second component P2. In some embodiments, the first component P1 is made of hard plastic or glass, and the second component P2 is made of foam plastic. In this way, the weight of the photon emission device 1220 can be reduced. In some embodiments, the third component P3 in FIG. 13C may have the same material as the second component P2.

In some embodiments, provided that the photons emitted by the photon emission source 1221 are capable of irradiating a predetermined target region, the shape, the size, the position of the photon emission source 1221, the first photon shielding structure 1222, the second photon shielding structure 1223, and the fixing structure 1224 can be adjusted as required. More descriptions regarding the target region may be found elsewhere in the present disclosure.

For example, as shown in FIG. 13F, the first photon shielding structure 1222 is an annular member having a concave structure (e.g., a cylindrical concave structure). The photon emission source 1221 is disposed within the concave structure. The photon emission source 1221 is configured as a cylindrical body. The second photon shielding structure 1223 is configured as a cylindrical body. The fixing structure 1224 includes an annular member (i.e., the first component P1) arranged peripherally surround the photon emission source 1221, and secures the positions of the photon emission source 1221 and the second photon shielding structure 1223 relative to the first photon shielding structure 1222. A first portion of the first component P1 is disposed within the concave structure and surrounds the photon emission source, and a second portion of the first component P1 is outside the concave structure and surrounds the second photon shielding structure. An outer diameter of the first component P1 is substantially equal to a diameter of the concave structure, an inner diameter of the first portion of the fixing structure 1224 surrounding the photon emission source 1221 is substantially equal to an outer diameter of the photon emission source 1221, and an inner diameter of the second portion of the fixing structure 1224 surrounding the second photon shielding structure 1223 is substantially equal to an outer diameter of the second photon shielding structure 1223. For example, if the cross-section views of the photon emission devices 1220 shown in FIG. 13A correspond to the photon emission device 1220 shown in FIG. 13F, as shown in FIG. 13A, a bottom surface of the photon emission source 1221 facing the first photon shielding structure 1222 is connected to the bottom surface CS1 of the concave structure CS. The inner surface of the first component P1 is connected to a cylindrical surface of the photon emission source 1221. A cylindrical surface of the first component P1 and a bottom surface of the first component P1 facing the first photon shielding structure 1222 are connected to the concave structure CS. A portion of the inner surface of the first component P1 surrounding the second photon shielding structure 1223 is connected to a cylindrical surface of the second photon shielding structure 1223 and a portion of a bottom surface of the second photon shielding structure 1223 facing the first photon shielding structure 1222. In some alternative embodiments, the inner diameter of the first portion of the fixing structure 1224 and the inner diameter of the second portion of the fixing structure 1224 are the same. In such cases, the diameters of the photon emission source 1221 and the second photon shielding structure 1223 are the same.

In some embodiments, the photon emission device 1220 is configured such that the photons irradiate the target region, and the target region covers the detector 1210 but does not cover the scanning object 1230. As used herein, the target region refers to a region that the photons emitted by the photon emission device 1220 can irradiate. By means of this configuration, additional radiation exposure to the scanning object 1230 induced by the photons emitted by the photon emission device 1220 can be avoided.

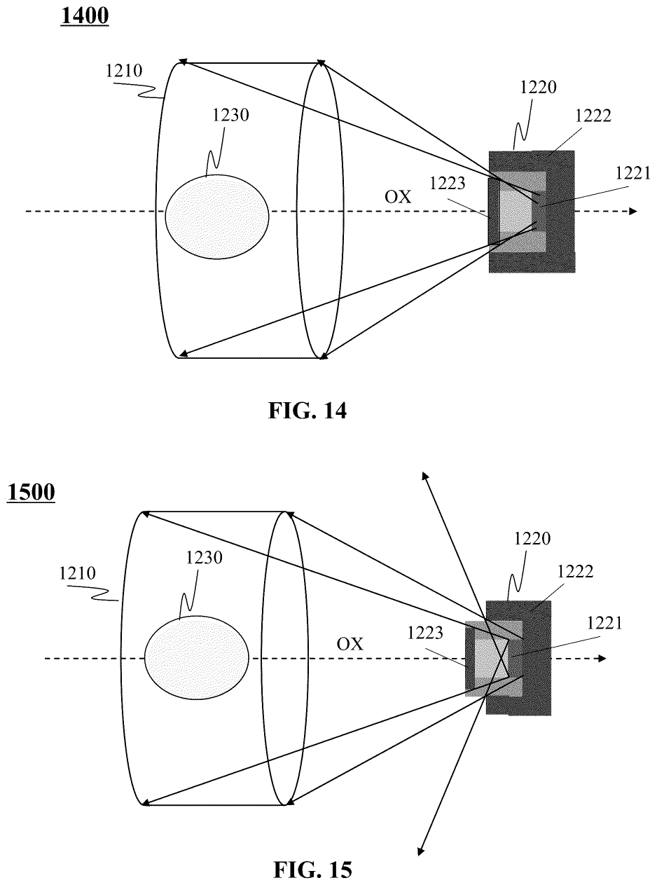

In some embodiments, the target region only covers the detector 1210 without covering any region outside the imaging field. For example, FIG. 14 is a schematic diagram illustrating an exemplary ECT scanner 1400 according to some embodiments of the present disclosure. As shown in FIG. 14, the target region only covers the detector 1210 without covering any region outside the imaging field. In some embodiments, the target region covers the detector 1210 and one or more regions outside the imaging field, but does not cover the target object 1230. For example, FIG. 15 is a schematic diagram illustrating an exemplary ECT scanner 1500 according to some embodiments of the present disclosure. As shown in FIG. 15, the target region covers the detector 1210 and one or more regions outside the imaging field.

A suitable target region may be determined by adjusting parameters such as sizes, shapes, and relative positions of the components (e.g., the photon emission source 1221, the first photon shielding structure 1222, the second photon shielding structure 1223, the fixing structure 122, etc.) of the photon emission device 1220. In some embodiments, the target region is determined by adjusting a relative position of the photon emission source and the first photon shielding structure or a relative position of the first photon shielding structure and the second photon shielding structure. Merly by way of example, as shown in FIG. 14 and FIG. 15, the distance between the first photon shielding structure 1222 and the second photon shielding structure 1223 corresponding to the ECT scanner 1400 is smaller than the distance between the first photon shielding structure 1222 and the second photon shielding structure 1223 corresponding to the ECT scanner 1500, the distance between the photon emission source 1221 and the second photon shielding structure 1223 corresponding to the ECT scanner 1400 is smaller than the distance between the photon emission source 1221 and the second photon shielding structure 1223 corresponding to the ECT scanner 1500. The target region corresponding to the ECT scanner 1400 is smaller than the target region corresponding to the ECT scanner 1500.

Using the ECT scanner 1200 introducing the photon emission device 1220, additional backscattering coincidence events are generated, thereby supplying more data for the reconstruction of the attenuation-corrected radiological image of the scanning object and ultimately improving the accuracy of attenuation correction.

Particularly in cases where the activity of the tracer in the scanning object is low and the number of backscattering coincidence events is limited, using the ECT scanner 1200, additional backscattering coincidence events generated via the photon emission device 1220 supplement more data for the reconstruction of the attenuation-corrected image of the scanning object, thereby ensuring the accuracy of attenuation correction.

The backscattering coincidence events obtained using the ECT scanner 1200 exhibit a broad angular distribution characteristic, enabling coverage the entire detector. This allows for effective coverage of the full imaging field of view (FOV) without the need for mechanical rotation or scanning, thereby generating stable and complete transmission information. At the same time, the use of a statically installed photon emission source eliminates the complex mechanical drive structures required in traditional rotating source schemes, reducing equipment cost and size, eliminating safety risks associated with moving parts, and enhancing the overall system stability and clinical operational convenience.

It is worth noting that the gamma photons generated by backscatter have relatively low energy. Based on typical parameter estimates, acquiring approximately 3×10⁸ backscattering events over a 10-minute scan in a short-axis system results in an equivalent dose of only 0.002 mSv, which is significantly lower than the radiation levels of conventional medical imaging. Therefore, the ECT scanner 1200 is particularly suitable for applications requiring repeated scans or for sensitive populations such as pediatric patients.

In terms of engineering implementation, the photon emission source of the ECT scanner 1200 can be designed as a fixed structure, installed on the gantry or housing without occupying imaging field space. Moreover, the photon emission source exhibits high radioactivity stability and slow attenuation, making it suitable for long-term continuous operation, thereby substantially reducing maintenance costs and operational complexity associated with frequent source replacement or replenishment.

FIG. 16 is a schematic diagram illustrating an exemplary ECT scanner 1600 according to some embodiments of the present disclosure. In some embodiments, the ECT scanner 1600 may be an exemplary embodiment of the scanning device 110 as described in connection with FIG. 1. The ECT scanner 1600 may be similar to the ECT scanner 1200 as described in connection with FIG. 12, except that the ECT scanner 1600 includes a pair of photon emission devices 1220.

As shown in FIG. 16, the pair of photon emission devices 1220 are symmetrically arranged relative to a central axis OX of the imaging field, and the pair of photon emission devices 1220 are spaced apart from the detector along the central axis.

Multi-bed position scanning in ECT imaging is designed to extend the axial field of view (FOV) beyond the physical limits of the detector of the ECT scanner. Usually, this technique is essential for whole-body imaging, allowing comprehensive coverage from the head to the feet. During a multi-bed position scan, a scanning table supporting the scanning object is required to move along the central axis OX of the imaging field several times to scan different portions of the scanning object. The ECT scanner 1600 is suitable for the multi-bed position scan, which can prevent collision between the photon emission devices 1220 and the scanning table.

It should be noted that the ECT scanners 1200 and 1600 illustrated in FIGS. 12 and 16 and the descriptions thereof are provided for the purposes of illustration, and not intended to limit the scope of the present disclosure. For persons having ordinary skills in the art, various modifications and changes in the forms and details of the application may occur without departing from the principles of the present disclosure. However, those variations and modifications also fall within the scope of the present disclosure. For example, the ECT scanners 1200 or 1600 may include multiple photon emission devices 1220. As another example, the positions of the one or more photon emission device 1220 of the ECT scanners 1200 or 1600 may be adjusted according to needs.

FIG. 17 is a flowchart illustrating an exemplary process 1700 for attenuation correction according to some embodiments of the present disclosure. The process 1700 is performed based on scan data collected using an ECT scanner (e.g., the ECT scanner 1200 or the ECT scanner 1600) disclosed herein. For illustration purposes, the implementation of the process 1700 based on scan data collected using the ECT scanner 1200 is described as an example.

In 1710, the processing device 120 (e.g., the first obtaining module 210) may obtain first scan data collected by the detector in a blank scan without a scanning object in the imaging field.

In some embodiments, a backscattering event is produced by Compton scattering of gamma photons within the detector. When the scattering angle exceeds 120 degrees, the scattered photons penetrate through the scanning object along the reverse path and are detected by the opposite detector.

As used herein, the blank scan refers to a scan performed using the ECT scanner 1200 when no scanning object is located in the imaging field. During the blank scan, the photon emission device 1220 emits photons, wherein at least a portion of these photons enter the imaging field formed by the detector and strike the detector. Part of the photons striking the detector may scatter, resulting in first backscattering coincidence events occur in the blank scan. A backscattering coincidence event refers to an event where two scattered photons are detected by different detector units of the detector within a coincidence time window corresponding to backscattering coincidence event detection.

In 1720, the processing device 120 (e.g., the first obtaining module 210 and the second obtaining module 210) may obtain second scan data collected by the detector (i.e., the detector 1210) in an ECT scan with the scanning object in the imaging field.

During the ECT scan, the photon emission device 1220 emits photons, wherein at least a portion of these photons enter the imaging field formed by the detector and strike the detector. Part of the photons striking the detector may scatter, resulting in second backscattering coincidence events occur in the ECT scan. Before the ECT scan, a tracer is introduced into the scanning object. During the ECT scan, photons generated by positive and negative electron annihilation coincidence events induced by the tracer may strike the detector, and part of the photons may scatter, resulting in second backscattering coincidence events occur in the ECT scan. The second backscattering coincidence events originating from the photon emission device 1220 are referred to as second backscattering coincidence events S1, and the second backscattering coincidence events originating from the positive and negative electron annihilation coincidence events in the scanning object are referred to as second backscattering coincidence events S2.

In some embodiments, scan data (e.g., the first scan data, the second scan data) collected by the detector 1210 may include data related to the photons collected by the detector 1210. Exemplary data related to the photons may include locations of detector units detecting the photons, energy information of the photons, detection times (also referred to as arrival times of the photons) when the photons are detected, or the like, or any combination thereof.

In 1730, the processing device 120 (e.g., the reconstruction module 230) may generate an attenuation-corrected ECT image of the scanning object based on the first scan data and the second scan data.

The attenuation-corrected ECT image is an exemplary embodiment of the attenuation-corrected radiological image as described elsewhere in this disclosure (e.g., FIG. 3 and the relevant descriptions).

In some embodiments, the processing device 120 may determine first backscattering coincidence event data based on the first scan data. The processing device 120 may also determine radiological coincidence event data and second backscattering coincidence event data based on the second scan data. Further, the processing device 120 may generate the attenuation-corrected ECT image of the scanning object based on the first backscattering coincidence event data, the second backscattering coincidence event data, and the radiological coincidence event data.

The first backscattering coincidence event data refers to data relating to the first backscattering coincidence events occur in the blank scan, wherein the first backscattering coincidence events are caused by the photon emission source 1220. The second backscattering coincidence event data refers to data relating to the second backscattering coincidence events occur in the ECT scan, wherein the second backscattering coincidence events are caused by the photon emission source 1220 and the positive and negative electron annihilation coincidence events in the scanning object. The second backscattering coincidence event data may be interpreted as the measured total backscattering coincidence event data during the ECT scan, which includes backscattering coincidence event data corresponding to the positive and negative electron annihilation coincidence events within the scanning object and backscattering coincidence event data corresponding to the photon emission source 1220.

As described in operation 320, data relating to backscattering coincidence events includes, for example, a count, a trajectory, etc., of the backscattering coincidence events. In some embodiments, the processing device 120 may determine the first backscattering coincidence event data and the second backscattering coincidence event data using the dual-coincidence strategy in a similar manner as how to determine the backscattering coincidence event data in the transmission data described in operation 320, and the descriptions of which are not repeated here.

The radiological coincidence event data refers to the data related to the radiological coincidence events (i.e., positive and negative electron annihilation coincidence events) occur in the ECT scan. For example, the data related to the radiological coincidence events may include a count, a trajectory, etc., of the radiological coincidence events of the scanning object. In some embodiments, the processing device 120 may determine the radiological coincidence event data in a similar manner as described in operation 310, and the descriptions of which are not repeated here.

To generate the attenuation-corrected ECT image of the scanning object, an operation similar to the process 600 (in which the operation 630 is performed by the process 700) is performed, except a difference that the blank scanning estimation for backscattering described the process 700 needs to be replaced by a total blank scanning estimation for backscattering (denoted as Blankscan_all). The total blank scanning estimation refers to a statistical distribution of backscattering coincidence events simulated under a blank scan condition—i.e., without any attenuating or scattering material other than the photon emission device. As described above, the second backscattering coincidence events include both the second backscattering coincidence events S1 and second backscattering coincidence events S2, and thus Blankscan_all includes both a blank scanning estimation for backscattering corresponding to the second backscattering coincidence events S1 originated from the photon emission device (also referred to as reference blank scanning estimation) and a blank scanning estimation for backscattering corresponding to the second backscattering coincidence events S2 originated from the annihilation photons emitted from the scanning object. The blank scanning estimation for backscattering corresponding to the second backscattering coincidence events S2 may be determined in a manner similar to the blank scanning estimation for backscattering described the process 700.

Since the location and activity distribution of the photon emission device 1210 remain constant, it is possible to determine the blank scanning estimation for backscattering (i.e., the reference blank scanning estimation) corresponding to the second backscattering coincidence events S1 based on the first backscattering coincidence event data. In some embodiments, the processing device 120 may determine the reference blank scanning estimation for backscattering corresponding to the photon emission device 1210 based on the first backscattering coincidence event data, a scan duration of the blank scan, and a scan duration of the ECT scan. The reference blank scanning estimation for backscattering refers to a statistical distribution of backscattering coincidence events simulated or measured under a blank scan condition—i.e., without any attenuating or scattering material other than the photon emission device. The reference blank scanning estimation for backscattering may be expressed in a form of a sinogram. For example, the processing device 120 may determine the reference blank scanning estimation according to Equation (5) as below:

$$Blankscan\_ref = \frac{T 2}{T 1} * blank\_external , \qquad ( 5 )$$

where, Blankscan_ref denotes the reference blank scanning estimation for backscattering, blank_external denotes the first backscattering coincidence event data, T1 denotes the scan duration of the blank scan, and T2 denotes the scan duration of the ECT scan.

Further, the processing device 120 may generate the attenuation-corrected ECT image of the scanning object based on the reference blank scanning estimation for backscattering, the second backscattering coincidence event data, and the radiological coincidence event data. In some embodiments, the processing device 120 may generate the attenuation-corrected ECT image by performing a first reconstruction process similar to the process 600, wherein a portion of operation 630 is implemented by the process 700. In the first reconstruction process, the transmission data or the backscattering coincidence event data described in FIG. 6 and FIG. 7 may be replaced by the second backscattering coincidence event data of the scanning object.

In some embodiments, the first reconstruction process is an iterative reconstruction process. In the process of iteratively reconstructing the attenuation-corrected ECT images, an initial attenuation image and an initial radiological image are obtained based on an initialization of attenuation images and an initialization of radiological images as iterative inputs for the first round of iteration; in each round of iteration, the iterative input is analyzed and reconstructed to obtain an iterative attenuation image and an iterative radiological image, which are used to update the iterative input of the next iteration. In some embodiments, the iterative reconstruction process is similar to process 700. In the iterative reconstruction process, the backscattering coincidence event data described in FIG. 7 may be replaced by the second backscattering coincidence event data of the scanning object and the blank scanning estimation for backscattering 714 described in FIG. 7 may be replaced by the total blank scanning estimation for backscattering.

The total blank scanning estimation for backscattering may be determined based on the reference blank scanning estimation for backscattering. For example, the processing device 120 may determine the total blank scanning estimation according to Equation (6) as below:

$$Blankscan\_all = Blankscan\_ref + {B}_{bs}^{k} , \qquad ( 6 )$$

where, Blankscan_all denotes the total blank scanning estimation, and

$${B}_{bs}^{k}$$

denotes the blank scanning estimation for backscattering described in operation 720 (i.e., the first portion of the total blank scanning estimation for backscattering).

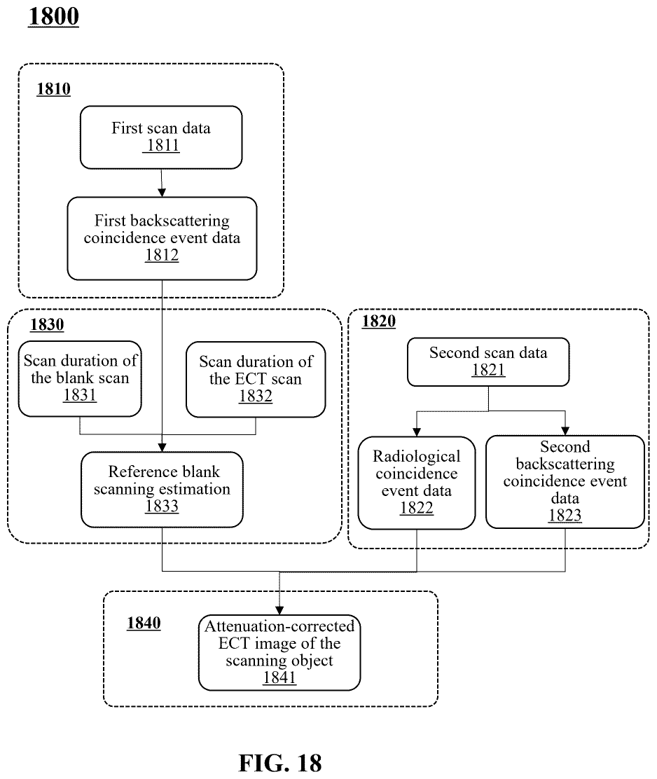

FIG. 18 is a schematic diagram illustrating an exemplary process 1800 for obtaining an attenuation-corrected ECT image according to some embodiments of the present disclosure. In some embodiments, one or more operations of the process 1800 may be performed to achieve operation 1730 as described in connection with FIG. 17.

In 1810, first backscattering coincidence event data 1812 may be determined based on first scan data 1811.

The first scan data 1811 may be collected by using the ECT scanner 1200 or the ECT scanner 1600 to perform a blank scan without a scanning object in the imaging field. The first backscattering coincidence event data is determined based on the first scan data using the dual-coincidence strategy.

In 1820, radiological coincidence event data 1822 and second backscattering coincidence event data 1823 may be determined based on second scan data 1821.

The second scan data 1821 may be collected by using the ECT scanner 1200 or the ECT scanner 1600 to perform an ECT scan with the scanning object in the imaging field.

More descriptions regarding the determination of the radiological coincidence event data and second backscattering coincidence event data based on the second scan data may be found elsewhere in the present disclosure. See, e.g., operation 1730 in FIG. 17 and relevant descriptions thereof.

In 1830, a reference blank scanning estimation for backscattering 1833 corresponding to the photon emission device 1210 may be determined based on the first backscattering coincidence event data 1812, a scan duration of the blank scan 1831, and a scan duration of the ECT scan 1832.

More descriptions regarding the determination of the reference blank scanning estimation for backscattering may be found elsewhere in the present disclosure. See, e.g., operation 1730 in FIG. 17 and relevant descriptions thereof.

In 1840, an attenuation-corrected ECT image 1841 of the scanning object may be generated based on the reference blank scanning estimation for backscattering 1833, the second backscattering coincidence event data 1823, and the radiological coincidence event data 1822.

More descriptions regarding the generation of the attenuation-corrected ECT image may be found elsewhere in the present disclosure. See, e.g., operation 1730 in FIG. 17 and relevant descriptions thereof.

In some occasions, an unknown photon emission source (e.g., the liver, the bladder, an injection site for injecting the tracer, or other organ or tissue with high activity) may present outside the imaging field of the scanning device 110. Some photons generated by annihilation events occurring within the unknown photon emission source (referred to as unknown photons for brevity) may be detected by the detector of the scanning device 110. Some of the unknown photons entered the detector may be scattered by the detector, subsequently traverse the scanning object to generate backscattering coincidence events. Backscattering coincidence event data determined using the dual-coincidence strategy (also referred to as double backscattering coincidence event data) relates to backscattering coincidence events originating from photons produced by annihilation events within the imaging field and backscattering coincidence events induced by the unknown photons. However, the backscattering coincidence events induced by the unknown photons may introduce noises, which may reduce the accuracy of the obtained double backscattering coincidence event data and consequently reduce the accuracy of the attenuation-corrected ECT image generated based on the double backscattering coincidence event data. As used herein, the double backscattering coincidence event data relates to backscattering coincidence events defined by two single events (i.e., backscattering coincidence events detected by the dual-coincidence strategy). The backscattering coincidence events defined by two single events are also referred to as double backscattering coincidence events. For example, the backscattering coincidence event data described in operation 320 is double backscattering coincidence event data, the two single events may be the single event 1 and the single event 2 described in operation 320.

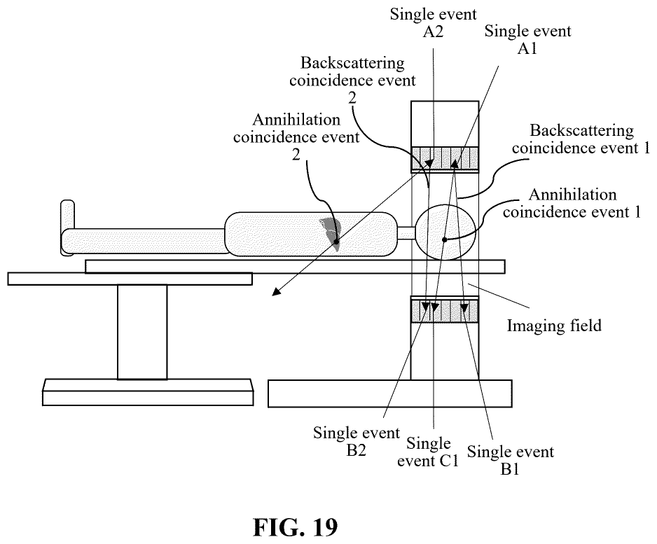

For example, FIG. 19 is a schematic diagram illustrating exemplary backscattering coincidence events generated by different photon emission sources according to some embodiments of the present disclosure. As shown in FIG. 19, an annihilation coincidence event 2 occurs in an unknown photon emission source outside the imaging field of the scanning device 110. One photon produced by the annihilation coincidence event 2 strikes the detector, undergoes Compton scattering, travels back through the scanning object, and strikes the detector again. In this process, two single events A2 and B2 are detected. When using the dual-coincidence strategy, a backscattering coincidence event 2 corresponding to the two single events A2 and B2 is determined, which may affect the accuracy of attenuation correction.

To address the above issues, the present disclosure provides an attenuation correction method based on a triple-coincidence strategy. The triple-coincidence strategy is used for determining backscattering coincidence events defined by three single events. The backscattering coincidence events defined by three single events are also referred to as triple backscattering coincidence events.

For example, as shown in FIG. 19, an annihilation coincidence event 1 occurs within the imaging field of the scanning device 110. The annihilation coincidence event 1 occurring within the imaging field produces two photons, one of the photons strikes by the detector, undergoes Compton scattering, travels back through the scanning object, and strikes the detector again. In this process, two single events A1 and B1 are detected. The other photon produced by the annihilation coincidence event 1 strikes the detector, and a single event C1 is detected. When using the triple-coincidence strategy, a backscattering coincidence event 1 corresponding to the single events A1, B1, and C1 is determined. When using the triple-coincidence strategy, since the other photon produced by the annihilation coincidence event 2 is not detected by the detector, the backscattering coincidence event corresponding to A2 and B2 is not wrongly detected.

Therefore, the attenuation correction methods based on the triple-coincidence strategy can eliminate or reduce the effect of backscattering coincidence events generated by the unknown photon emission source outside the imaging field, thereby improving the accuracy of the obtained backscattering coincidence event data for attenuation correction and consequently improving the accuracy of the attenuation-corrected ECT image generated based on the backscattering coincidence event data.

FIG. 2000 is a flowchart illustrating an exemplary process 2000 for attenuation correction based on a triple-coincidence strategy according to some embodiments of the present disclosure.

In 2010, the processing device 120 (e.g., the first obtaining module 210 and the second obtaining module 210) may obtain scan data of a scanning object collected by an ECT scanner.

The ECT scanner may be the scanning device 110. The ECT scan is performed after a tracer is introduced into the scanning object.

More descriptions regarding the scan data may be found elsewhere in the present disclosure (e.g., FIG. 17 and the descriptions thereof). The processing device 120 may obtain the scan data from one or more components (e.g., the scanning device 110, the storage device 130, etc.) of the system 100 or an external source via a network (e.g., the network 150).

In 2020, the processing device 120 (e.g., the determination module 240) may determine, based on the scan data, radiological coincidence event data and triple backscattering coincidence event data, wherein the triple backscattering coincidence event data relates to backscattering coincidence events defined by three single events (i.e., triple backscattering coincidence events).

The processing device 120 may determine the radiological coincidence event data in a similar manner as described in operation 310, and the descriptions of which are not repeated here.

In some embodiments, the three single events correspond to a first energy window, a second energy window, and a third energy window, respectively. In some embodiments, the first energy window covers 340 keV, the second energy window covers 170 keV, and the third energy window covers 511 keV. In some embodiments, the first energy window is 250-380 keV, the second energy window is 140-250 keV, and the third energy window is 430-650 keV. The first energy window, the second energy window, and the third energy window are determined in a variety of manners. For example, the first energy window, the second energy window, or the third energy window are determined in similar manners as the energy windows (e.g., the third preset energy window, the third preset energy window, etc.) described elsewhere in the present disclosure.

The processing device 120 may determine the triple backscattering coincidence event data based on the scan data and a coincidence time window (also referred to as a prompt window). Specifically, whenever one single event of the three single events is detected, the processing device 120 determines a time window starting from an arrival time of a photon corresponding to the single event as the prompt window. Further, the processing device 120 may determine whether remaining two single events of the three single events are detected within the prompt window based on the scan data. In response to determining that the remaining two single events of the three single events are detected within this prompt window, the processing device 120 determines that the three single events are identified as a triple backscattering coincidence event. An duration of the prompt window may be set according to experience or needs. For example, the duration of the prompt window is 4.4 nanoseconds, 9.9 nanoseconds, etc.

For brief, among the three single events, the single event corresponding to the first energy window is referred to as a first single event, the single event corresponding to the second energy window is referred to as a second single event, and the single event corresponding to the third energy window is referred to as a third single event.

For example, each time the first single event is detected, the processing device 120 determines a prompt window starting from an arrival time of a photon corresponding to the first single event. Further, the processing device 120 may determine whether the second and third single events are detected within the prompt window based on the scan data. In response to determining that the second and third single events are detected within this prompt window, the processing device 120 determines that the three single events are identified as a triple backscattering coincidence event.

As another example, each time the second single event is detected, the processing device 120 determines a prompt window starting from an arrival time of a photon corresponding to the second single event. Further, the processing device 120 may determine whether the first and third single events are detected within the prompt window based on the scan data. In response to determining that the first and third single events are detected within this prompt window, the processing device 120 determines that the three single events are identified as a triple backscattering coincidence event.

As still another example, each time the third single event is detected, the processing device 120 determines a prompt window starting from an arrival time of a photon corresponding to the third single event. Further, the processing device 120 may determine whether the first and second single events are detected within the prompt window based on the scan data. In response to determining that the first and second single events are detected within this prompt window, the processing device 120 determines that the three single events are identified as a triple backscattering coincidence event.

In 2030, the processing device 120 (e.g., the reconstruction module 230) may generate an attenuation-corrected ECT image of the scanning object based on the triple backscattering coincidence event data and the radiological coincidence event data.

In some embodiments, the processing device 120 generates the attenuation-corrected ECT image of the scanning object using the triple backscattering coincidence event data and the radiological coincidence event data by performing a second reconstruction process similar to a process for reconstructing the attenuation-corrected radiological image (e.g., the attenuation-corrected radiological image 742) described in FIG. 6. In the second reconstruction process, the transmission data or the backscattering coincidence event data of the target object described in FIG. 6 may be replaced by the triple backscattering coincidence event data of the scanning object.

Since each triple backscattering coincidence event involves three single events, the triple backscattering coincidence event data may include more complex random triple backscattering coincidence events. For example, there may be four kinds of random triple backscattering coincidence events, which are referred to as first random triple backscattering coincidence events, second random triple backscattering coincidence events, third random triple backscattering coincidence events, and fourth random triple backscattering coincidence events herein. In a first random triple backscattering coincidence event, two photons corresponding to the second single event and the third single event are originated from the same annihilation event, while the photon corresponding to the first single event is originated from another annihilation event. In a second triple backscattering random coincidence event, two photons corresponding to the first single event and the third single event are originated from the same annihilation event, while the photon corresponding to the second single event is originated from another annihilation event. In a third random triple backscattering coincidence event, two photons corresponding to the first single event and the second single event are originated from the same annihilation event, while the photon corresponding to the third single event is originated from another annihilation event. In a fourth random triple backscattering coincidence event, the photons corresponding to the first single event, the second single event, and the third single event are originated from three different annihilation events.

To reduce or eliminate the effect of the random triple backscattering coincidence events on the attenuation correction, in some embodiments, a dual delay window random correction strategy is adopted. The dual delay window random correction strategy utilizes a first delay window and a second delay window to determine the random triple backscattering coincidence events. Specifically, the processing device 120 may determine random event estimation for the triple backscattering coincidence events based on the scan data, the first delay window, and the second delay window. Further, the processing device 120 may generate the attenuation-corrected ECT image of the scanning object based on the triple backscattering coincidence event data, the radiological coincidence event data, and the random event estimation. In the reconstruction process of the attenuation-corrected ECT image, attenuation correction is performed based on the triple backscattering coincidence event data, and random correction is performed based on the random event estimation.

In some embodiments, each time a single event is detected, the processing device 120 determines a prompt window using an arrival time of a photon corresponding to the single event as the starting time of the prompt window to determine whether there is a triple backscattering coincidence event. The processing device 120 also determines a first delay window and a second delay window based on the prompt window to determine whether there is a random triple backscattering coincidence event.

In some embodiments, the first delay window, the second delay window, and the prompt window for determining the triple backscattering coincidence events are not overlapped. Specifically, the processing device 120 may determine a time window after the prompt window as the first delay window. The processing device 120 may also determine a time window after the first delay window as the second delay window.

Durations of the first delay window and the second delay window may be set according to experience or needs, for example, 4.4 nanoseconds, 9.9 nanoseconds, or the like. In some embodiments, durations of the prompt window, the first delay window, and the second preset window may be the same. Alternatively, durations of the prompt window, the first delay window, and the second preset window may be different. In some embodiments, an interval between the prompt window and the first delay window is greater than a first preset time threshold. In some embodiments, an interval between the first delay window and the second delay window is greater than a second preset time threshold. The first preset time threshold and the second preset time threshold may be set according to experience or needs. For example, the first preset time threshold is 100 nanoseconds, 150 nanoseconds, etc. The second preset time is threshold 50 nanoseconds, 100 nanoseconds, etc.

In some embodiments, the processing device 120 may determine first random event estimation, second random event estimation, and third random event estimation based on the scan data and the first delay window. The processing device 120 may also determine fourth random event estimation based on the first delay window, the second delay window, and the scan data. Then, the processing device 120 may determine the random event estimation based on the first random event estimation, the second random event estimation, the third random event estimation, and the fourth random event estimation.

The first random event estimation relates to a first random photon corresponding to the first energy window. The first random photon deposits energy in the first energy window. The first random event estimation may be used to estimate random triple backscattering coincidence events in which the photon corresponding to the first single event is a random photon (i.e., the first random photon). The first random event estimation may be used to estimate the first random triple backscattering coincidence events and the fourth random triple backscattering coincidence events.

In some embodiments, each time a first single event is detected, the processing device 120 may determine a prompt window and a first delay window corresponding to the first single event, and each time a third single event is detected, the processing device 120 may determine a prompt window and a first delay window corresponding to the third single event. Further, the processing device 120 may determine the first random event estimation based on the scan data, the prompt windows and the first delay windows corresponding to the first single event and the third single event. More descriptions regarding the determination of the prompt window and the first delay window may be found elsewhere in the present disclosure.

Specifically, each time a first single event is detected, the processing device 120 may determine whether a second single event and a third single event are detected within the first delay window corresponding to the first single event based on the scan data. In response to determining that the second single event and the third single event are detected within the first delay window corresponding to the first single event, the processing device 120 determines that the three single events are identified as a random triple backscattering coincidence event in the first random event estimation. Each time a third single event is detected, the processing device 120 may determine whether a second single event is detected within the prompt window corresponding to the third single event and a first single event is detected within the first delay window corresponding to the third single event based on the scan data. In response to determining that the second single event is detected within the prompt window corresponding to the third single event and the first single event is detected within the first delay window corresponding to the third single event, the processing device 120 determines that the three single events are identified as a random triple backscattering coincidence event in the first random event estimation.

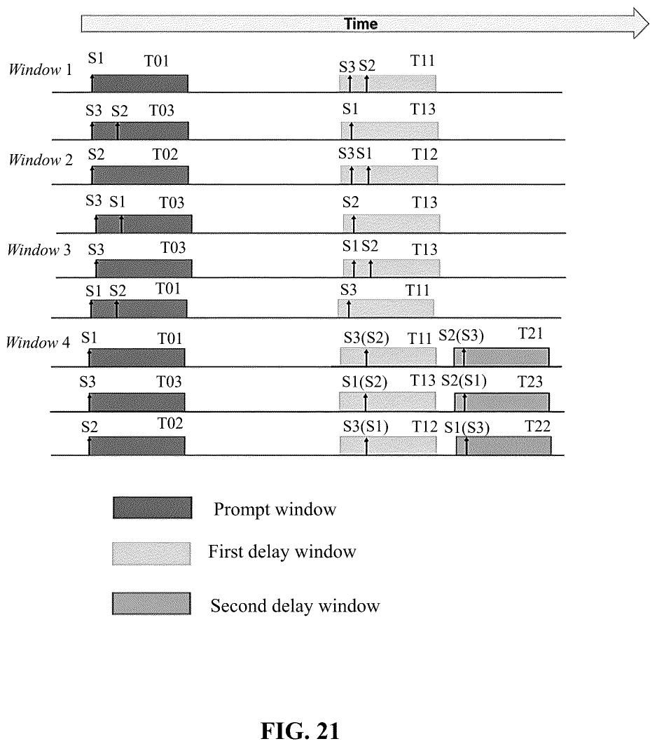

For example, FIG. 21 is a schematic diagram illustrating an exemplary process for determining random event estimation for triple backscattering coincidence events according to some embodiments of the present disclosure. As shown in FIG. 21, the processing device 120 may determine first random event estimation Window 1 based on the scan data. Specifically, each time a first single event S1 is detected, the processing device 120 may determine a prompt window T01 and a first delay window T11 corresponding to the first single event S1. Further, the processing device 120 may determine whether a second single event S2 and a third single event S3 are detected within the first delay window T11 based on the scan data. In response to determining that the second single event S2 and the third single event S3 are detected within the first delay window T11, the processing device 120 determines that the three single events S1-S3 are identified as a random triple backscattering coincidence event in the first random event estimation Window 1. Each time a third single event S3 is detected, the processing device 120 may determine a prompt window T03 and a first delay window T13 corresponding to the third single event S3. Further, the processing device 120 may determine whether a second single event S2 is detected within the prompt window T03 and a first single event S1 is detected within the first delay window T13 based on the scan data. In response to determining that the second single event S2 is detected within the prompt window T03 and the first single event S1 is detected within the first delay window T13, the processing device 120 determines that the three single events S1-S3 are identified as a random triple backscattering coincidence event in the first random event estimation Window 1.

The second random event estimation relates to a second random photon corresponding to the second energy window. The second random photon deposits energy in the second energy window. The second random event estimation may be used to estimate random triple backscattering coincidence events in which the photon corresponding to the second single event is a random photon (i.e., the second random photon). The second random event estimation may be used to estimate the second random triple backscattering coincidence events and the fourth random triple backscattering coincidence events.

In some embodiments, each time a second single event is detected, the processing device 120 may determine a prompt window and a first delay window corresponding to the second single event, and each time a third single event is detected, the processing device 120 may determine a prompt window and a first delay window corresponding to the third single event. Further, the processing device 120 may determine the second random event estimation based on the scan data, the prompt windows and the first delay windows corresponding to the second single event and the third single event. More descriptions regarding the determination of the prompt window and the first delay window may be found elsewhere in the present disclosure.

Specifically, each time a second single event is detected, the processing device 120 may determine whether a first single event and a third single event are detected within the first delay window corresponding to the second single event based on the scan data. In response to determining that the first single event and the third single event are detected within the first delay window corresponding to the second single event, the processing device 120 determines that the three single events are identified as a random triple backscattering coincidence event in the second random event estimation. Each time a third single event is detected, the processing device 120 may determine whether a first single event is detected within the prompt window corresponding to the third single event and a second single event is detected within the first delay window corresponding to the third single event based on the scan data. In response to determining that the first single event is detected within the prompt window corresponding to the third single event and the second single event is detected within the first delay window corresponding to the third single event, the processing device 120 determines that the three single events are identified as a random triple backscattering coincidence event in the second random event estimation.

For example, as shown in FIG. 21, the processing device 120 may determine second random event estimation Window 2 based on the scan data. Specifically, each time a second single event S2 is detected, the processing device 120 may determine a prompt window T02 and a first delay window T12 corresponding to the second single event S2. Further, the processing device 120 may determine whether a first single event S1 and a third single event S3 are detected within the first delay window T12 based on the scan data. In response to determining that the first single event S1 and the third single event S3 are detected within the first delay window T12, the processing device 120 determines that the three single events S1-S3 are identified as a random triple backscattering coincidence event in the second random event estimation Window 2. Each time a third single event S3 is detected, the processing device 120 may determine a prompt window T03 and a first delay window T13 corresponding to the third single event S3. Further, the processing device 120 may determine whether a first single event S1 is detected within the prompt window T03 and a second single event S2 is detected within the first delay window T13 based on the scan data. In response to determining that the first single event S1 is detected within the prompt window T03 and the second single event S2 is detected within the first delay window T13, the processing device 120 determines that the three single events S1-S3 are identified as a random triple backscattering coincidence event in the second random event estimation Window 2.

The third random event estimation relates to a third random photon corresponding to the third energy window. The third random photon deposits energy in the third energy window. The third random event estimation may be used to estimate random triple backscattering coincidence events in which the photon corresponding to the third single event is a random photon (i.e., the third random photon). The third random event estimation may be used to estimate the third random triple backscattering coincidence events and the fourth random triple backscattering coincidence events.

In some embodiments, each time a third single event is detected, the processing device 120 may determine a prompt window and a first delay window corresponding to the third single event, and each time a first single event is detected, the processing device 120 may determine a prompt window and a first delay window corresponding to the first single event. Further, the processing device 120 may determine the third random event estimation based on the scan data, the prompt windows and the first delay windows corresponding to the first single event and the third single event. More descriptions regarding the determination of the prompt window and the first delay window may be found elsewhere in the present disclosure.

Specifically, each time a third single event is detected, the processing device 120 may determine whether a second single event and a first single event are detected within the first delay window corresponding to the third single event based on the scan data. In response to determining that the second single event and the first single event are detected within the first delay window corresponding to the third single event, the processing device 120 determines that the three single events are identified as a random triple backscattering coincidence event in the third random event estimation. Each time a first single event is detected, the processing device 120 may determine whether a second single event is detected within the prompt window corresponding to the first single event and a third single event is detected within the first delay window corresponding to the first single event based on the scan data. In response to determining that the second single event is detected within the prompt window corresponding to the first single event and the third single event is detected within the first delay window corresponding to the first single event, the processing device 120 determines that the three single events are identified as a random triple backscattering coincidence event in the third random event estimation.

For example, as shown in FIG. 21, the processing device 120 may determine third random event estimation Window 3 based on the scan data. Specifically, each time a third single event S3 is detected, the processing device 120 may determine a prompt window T03 and a first delay window T13 corresponding to the third single event S3. Further, the processing device 120 may determine whether a second single event S2 and a first single event S1 are detected within the first delay window T13 based on the scan data. In response to determining that the second single event S2 and the first single event S1 are detected within the first delay window T13, the processing device 120 determines that the three single events S1-S3 are identified as a random triple backscattering coincidence event in the third random event estimation Window 3. Each time a first single event S1 is detected, the processing device 120 may determine a prompt window T01 and a first delay window T11 corresponding to the first single event S1. Further, the processing device 120 may determine whether a second single event S2 is detected within the prompt window T01 and a third single event S3 is detected within the first delay window T11 based on the scan data. In response to determining that the second single event S2 is detected within the prompt window T01 and the third single event S3 is detected within the first delay window T11, the processing device 120 determines that the three single events S1-S3 are identified as a random triple backscattering coincidence event in the third random event estimation Window 3.

The fourth random event estimation relates to the first random photon, the second random photon, and the third random photon. The fourth random event estimation may be used to estimate random triple backscattering coincidence events in which the three photons corresponding to the first single event, the second single event, and the third single event are random photons. The fourth random event estimation may be used to estimate the fourth random triple backscattering coincidence events.

In some embodiments, each time a first single event is detected, the processing device 120 may determine a prompt window, a first delay window, and a second delay window corresponding to the first single event. Each time a second single event is detected, the processing device 120 may determine a prompt window, a first delay window, and a second delay window corresponding to the second single event. Each time a third single event is detected, the processing device 120 may determine a prompt window, a first delay window, and a second delay window corresponding to the third single event. Further, the processing device 120 may determine the fourth random event estimation based on the scan data, the prompt windows, the first delay windows, and the second delay windows corresponding to the first single event, the second single event, and the third single event. More descriptions regarding the determination of the prompt window, the first delay window, and the second delay window may be found elsewhere in the present disclosure.

Specifically, each time a first single event is detected, the processing device 120 may determine whether one of a second single event and a third single event is detected within the first delay window corresponding to the first single event, and another of the second single event and the third single event is detected within the second delay window corresponding to the first single event based on the scan data. In response to determining that one of the second single event and the third single event is detected within the first delay window corresponding to the first single event, and another of the second single event and the third single event is detected within the second delay window corresponding to the first single event, the processing device 120 determines that the three single events are identified as a random triple backscattering coincidence event in the fourth random event estimation. Each time a second single event is detected, the processing device 120 may determine whether one of a first single event and a third single event is detected within the first delay window corresponding to the second single event, and another of the first single event and the third single event is detected within the second delay window corresponding to the second single event based on the scan data. In response to determining that one of the first single event and the third single event is detected within the first delay window corresponding to the second single event, and another of the first single event and the third single event is detected within the second delay window corresponding to the second single event, the processing device 120 determines that the three single events are identified as a random triple backscattering coincidence event in the fourth random event estimation. Each time a third single event is detected, the processing device 120 may determine whether one of a second single event and a first single event is detected within the first delay window corresponding to the third single event, and another of the second single event and the first single event is detected within the second delay window corresponding to the third single event based on the scan data. In response to determining that one of the second single event and the first single event is detected within the first delay window corresponding to the third single event, and another of the second single event and the first single event is detected within the second delay window corresponding to the third single event, the processing device 120 determines that the three single events are identified as a random triple backscattering coincidence event in the fourth random event estimation.

For example, as shown in FIG. 21, the processing device 120 may determine fourth random event estimation Window 4 based on the scan data. Specifically, each time a first single event S1 is detected, the processing device 120 may determine a prompt window T01, a first delay window T11, and a second delay window T21 corresponding to the first single event S1. Further, the processing device 120 may determine whether one of a second single event S2 and a third single event S3 is detected within the first delay window T11 and another of the second single event S2 and the third single event S3 is detected within the second delay window T21 based on the scan data. In response to determining that one of the second single event S2 and the third single event S3 is detected within the first delay window T11 and another of the second single event S2 and the third single event S3 is detected within the second delay window T21, the processing device 120 determines that the three single events S1-S3 are identified as a random triple backscattering coincidence event in the fourth random event estimation Window 4. Each time a second single event S2 is detected, the processing device 120 may determine a prompt window T02, a first delay window T12, and a second delay window T22 corresponding to the second single event S2. Further, the processing device 120 may determine whether one of a first single event S1 and a third single event S3 is detected within the first delay window T12 and another of the first single event S1 and the third single event S3 is detected within the second delay window T22 based on the scan data. In response to determining that one of the first single event S1 and the third single event S3 is detected within the first delay window T12 and another of the first single event S1 and the third single event S3 is detected within the second delay window T22, the processing device 120 determines that the three single events S1-S3 are identified as a random triple backscattering coincidence event in the fourth random event estimation Window 4. Each time a third single event S3 is detected, the processing device 120 may determine a prompt window T03, a first delay window T13, and a second delay window T23 corresponding to the third single event S3. Further, the processing device 120 may determine whether one of a second single event S2 and a first single event S1 is detected within the first delay window T13 and another of the second single event S2 and the first single event S1 is detected within the second delay window T23 based on the scan data. In response to determining that one of the second single event S2 and the first single event S1 is detected within the first delay window T13 and another of the second single event S2 and the first single event S1 is detected within the second delay window T23, the processing device 120 determines that the three single events S1-S3 are identified as a random triple backscattering coincidence event in the fourth random event estimation Window 4.

The processing device 120 may determine the random event estimation based on the first random event estimation, the second random event estimation, the third random event estimation, and the fourth random event estimation. In some embodiments, the processing device 120 may determine a sum of the first random event estimation, the second random event estimation, and the third random event estimation. Further, the processing device 120 may determine the random event estimation by subtracting twice the fourth random event estimation from the sum of the first random event estimation, the second random event estimation, and the third random event estimation. For example, for the example shown in FIG. 21, the processing device 120 may determine the random event estimation according to Equation (7) as below:

$${R}_{random} = Window 1 + Window 2 + Window 3 - 2 Window 4 , \qquad ( 7 )$$

where, R_{random} denotes the random event estimation.

Further, the processing device 120 may generate the attenuation-corrected ECT image of the scanning object based on the triple backscattering coincidence event data, the radiological coincidence event data, and the random event estimation.

In some embodiments, the processing device 120 may correct the radiological coincidence event data to generate corrected radiological coincidence event data based on the random event estimation. For example, the processing device 120 may subtract the random event estimation from the radiological coincidence event data to generate the corrected radiological coincidence event data. Further, the processing device 120 may generate the attenuation-corrected ECT image of the scanning object using the triple backscattering coincidence event data and the corrected radiological coincidence event data by performing the second reconstruction process.

In some embodiments, the processing device 120 may generate the attenuation-corrected ECT image by performing a third reconstruction process similar to a process for reconstructing the attenuation-corrected radiological image (e.g., the attenuation-corrected radiological image 742) described in FIG. 6, wherein a portion of operation 630 is implemented by the process 700. In the third reconstruction process, the transmission data or the backscattering coincidence event data described in FIG. 6 and FIG. 7 may be replaced by the triple backscattering coincidence event data.

In some embodiments, the third reconstruction process is an iterative reconstruction process. In the process of iteratively reconstructing the attenuation-corrected ECT images, an initial attenuation image and an initial radiological image are obtained based on an initialization of attenuation images and an initialization of radiological images as iterative inputs for the first round of iteration; in each round of iteration, the iterative input is analyzed and reconstructed to obtain an iterative attenuation image and an iterative radiological image, which are used to update the iterative input of the next iteration. In some embodiments, the iterative reconstruction process is similar to process 700. In the iterative reconstruction process, the backscattering coincidence event data described in FIG. 7 may be replaced by the triple backscattering coincidence event data of the scanning object and the random event estimation for backscattering described in FIG. 7 may be replaced by the random event estimation for the triple backscattering coincidence events.

In some embodiments, for an ECT scan, whether there is an unknown photon emission source outside an imaging field of the ECT scanner may be determined before the ECT scan. If it is determined that there is no unknown photon emission source outside the imaging field, the dual-coincidence strategy or a combination of the dual-coincidence strategy and the triple-coincidence strategy is more suitable for the ECT scan because the dual-coincidence strategy or the combination strategy can determine more backscattering coincidence event data, thus achieving better attenuation correction accuracy and reconstruction. If it is determined that there is an unknown photon emission source outside the imaging field, the triple-coincidence strategy is more suitable for the ECT scan to reduce or eliminate the effect of the photons originated from the unknown photon emission source.

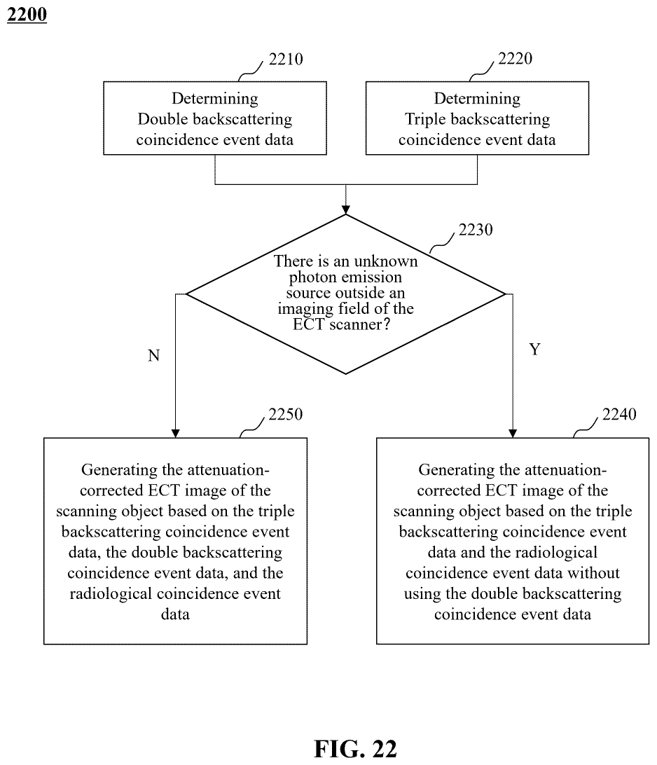

In some embodiments, the processing device 120 may determine whether there is an unknown photon emission source outside the imaging field of the ECT scanner by data analysis. For example, the processing device 120 may determine double backscattering coincidence event data relating to backscattering coincidence events defined by two single events (i.e., double backscattering coincidence events). Further, the processing device 120 may determine whether there is an unknown photon emission source outside the imaging field of the ECT scanner based on the double backscattering coincidence event data and the triple backscattering coincidence event data. In response to determining that there is no unknown photon emission source, the processing device 120 may generate the attenuation-corrected ECT image of the scanning object based on the triple backscattering coincidence event data, the double backscattering coincidence event data, and the radiological coincidence event data. In response to determining that there is an unknown photon emission source, the processing device 120 may generate the attenuation-corrected ECT image of the scanning object based on the triple backscattering coincidence event data and the radiological coincidence event data without using the double backscattering coincidence event data. More descriptions regarding the generation of the attenuation-corrected ECT image based on the radiological coincidence event data, the triple backscattering coincidence event data, or the double backscattering coincidence event data may be found elsewhere in the present disclosure (e.g., FIG. 22 and thereof descriptions).

In some embodiments, the amount of the triple backscattering coincidence event data (also referred to as first triple backscattering coincidence event data) determined in operation 2020 is relatively small, the process 1700 may be performed on the scanning subject except a difference that the second backscattering coincidence event data needs to be replaced by fourth backscattering coincidence event data. The fourth backscattering coincidence event data may be determined based on the scan data obtained in operation 2010 and the second scan data obtained in operation 1720. Specifically, the processing device 120 may determine first triple backscattering coincidence event data and first double backscattering coincidence event data based on the scan data obtained in operation 2010. Further, the processing device 120 may determine second triple backscattering coincidence event data and second double backscattering coincidence event data based on the second scan data obtained in operation 1720. Then, the processing device 120 may determine the fourth backscattering coincidence event data based on the first triple backscattering coincidence event data, first double backscattering coincidence event data, the second triple backscattering coincidence event data, and the second double backscattering coincidence event data. For example, the processing device 120 may determine fifth backscattering coincidence event data (i.e., the first double backscattering coincidence event data) based on the scan data obtained in operation 2010 using the dual-coincidence strategy. The processing device 120 may also determine sixth backscattering coincidence event data (i.e., the second double backscattering coincidence event data) based on the second scan data obtained in operation 1720 using the dual-coincidence strategy. Then, the processing device 120 may subtract the fifth backscattering coincidence event data from the sixth backscattering coincidence event data to generate the seven backscattering coincidence event data. The processing device 120 may also determine eighth backscattering coincidence event data (i.e., the second triple backscattering coincidence event data) based on the second scan data obtained in operation 1720 using the triple-coincidence strategy. The processing device 120 may determine a sum of the first triple backscattering coincidence event data, the seven backscattering coincidence event data, and the eighth backscattering coincidence event data as the fourth backscattering coincidence event data.

FIG. 22 is a flowchart illustrating an exemplary process 2200 for attenuation correction according to some embodiments of the present disclosure. In some embodiments, one or more operations of the process 2200 may be performed to achieve at least part of operation 2030 as described in connection with FIG. 20.

In 2210, the processing device 120 may determine double backscattering coincidence event data based on the scan data.

As described elsewhere in the present, the double backscattering coincidence event data relates to backscattering coincidence events defined by two single events and is determined by the dual-coincidence strategy. More descriptions regarding the determination of the double backscattering coincidence event data may be found elsewhere in the present disclosure (e.g., FIG. 3 and thereof descriptions).

In 2220, the processing device 120 may determine triple backscattering coincidence event data based on the scan data.

As described elsewhere in the present, the triple backscattering coincidence event data relates to backscattering coincidence events defined by three single events and is determined by the triple-coincidence strategy. More descriptions regarding the determination of the triple backscattering coincidence event data may be found elsewhere in the present disclosure (e.g., operation 2020 in FIG. 20 and thereof descriptions).

In 2230, the processing device 120 may determine whether there is an unknown photon emission source outside an imaging field of the ECT scanner based on the double backscattering coincidence event data and the triple backscattering coincidence event data.

In some embodiments, the processing device 120 may generate a first attenuation-corrected ECT image of the scanning object based on the triple backscattering coincidence event data and the radiological coincidence event data. The first attenuation-corrected ECT image of the scanning object is generated by performing the second reconstruction process or the third reconstruction process as described in connection with operation 2030 in FIG. 20.

The processing device 120 may also generate a second attenuation-corrected ECT image of the scanning object based on the double backscattering coincidence event data and the radiological coincidence event data. The second attenuation-corrected ECT image may be generated by performing the process 600 in FIG. 6.

Further, the processing device 120 may determine whether there is an unknown photon emission source outside the imaging field of the ECT scanner based on a difference between the first attenuation-corrected ECT image and the second attenuation-corrected ECT image. In response to determining that the difference between the first attenuation-corrected ECT image and the second attenuation-corrected ECT image is greater than a threshold, the processing device 120 may determine that there is an unknown photon emission source outside the imaging field of the ECT scanner, and operation 2240 may be performed. In response to determine that the difference between the first attenuation-corrected ECT image and the second attenuation-corrected ECT image is not greater than the threshold, the processing device 120 may determine that there is no unknown photon emission source outside the imaging field of the ECT scanner, and operation 2250 may be performed.

In 2240, the processing device 120 may generate the attenuation-corrected ECT image of the scanning object based on the triple backscattering coincidence event data and the radiological coincidence event data without using the double backscattering coincidence event data.

In some embodiments, the processing device 120 may directly designate the first attenuation-corrected ECT image as the attenuation-corrected ECT image of the scanning object.

In 2250, the processing device 120 may generate the attenuation-corrected ECT image of the scanning object based on the triple backscattering coincidence event data, the double backscattering coincidence event data, and the radiological coincidence event data.

In some embodiments, the processing device 120 may perform a fourth reconstruction process similar to a process for reconstructing the attenuation-corrected radiological image (e.g., the attenuation-corrected radiological image 742) described in FIG. 6. In the fourth reconstruction process, the transmission data or the backscattering coincidence event data of the target object described in FIG. 6 may be replaced by the triple backscattering coincidence event data and the double backscattering coincidence event data of the scanning object. In the fourth reconstruction process, both the double backscattering coincidence event data and the triple backscattering coincidence event data are used for attenuation correction, thus providing more data to estimate a more accurate attenuation map and improving the reconstruction accuracy.

In some embodiments, the processing device 120 may designate the second attenuation-corrected ECT image as the attenuation-corrected ECT image of the scanning object.

According to process 2200, after it is determined whether there is an unknown photon emission source outside the imaging field of the ECT scanner, an attenuation correction strategy more suitable for the ECT scan is selected according to the determination result. In this way, the accuracy of the obtained attenuation-corrected ECT image can be greatly improved.

It should be noted that the processes 1700, 2000, and 2200 and the descriptions thereof are provided for the purposes of illustration, and not intended to limit the scope of the present disclosure. For persons having ordinary skills in the art, various modifications and changes in the forms and details of the application of the above method and system may occur without departing from the principles of the present disclosure. However, those variations and modifications also fall within the scope of the present disclosure. For example, the operations of the illustrated processes 1700, 2000, and 2200 are intended to be illustrative. In some embodiments, the processes 1700, 2000, and 2200 may be accomplished with one or more additional operations not described, and/or without one or more of the operations discussed. Additionally, the order in which the operations of the processes 1700, 2000, and 2200 and regarding descriptions are not intended to be limiting. In some embodiments, backscattering coincidence event data generated by the photon emission device and lutetium background radiation event data can be jointly estimated. By combining these two sources of transmission data, the signal-to-noise ratio (SNR) can be effectively improved, enhancing the accuracy and stability of the attenuation correction.

One or more embodiments of the present disclosure further provide a computer-readable storage medium storing computer instructions, and when the computer reads the computer instructions in the storage medium, the computer performs the method for attenuation correction as described in any one of the above embodiments.

The basic concepts have been described above, and it is apparent to those skilled in the art that the foregoing detailed disclosure is intended as an example only and does not constitute a limitation of the present disclosure. While not expressly stated herein, those skilled in the art may make various modifications, improvements, and amendments to the present disclosure. Those types of modifications, improvements, and amendments are suggested in the present disclosure, so these types of modifications, improvements, and amendments remain within the spirit and scope of the exemplary embodiments of the present disclosure.

Also, the present disclosure uses specific words to describe embodiments thereof. Such as “an embodiment,” “one embodiment,” and/or “some embodiments” means a feature, structure, or characteristic associated with at least one embodiment of the present disclosure. Accordingly, it should be emphasized and noted that “one embodiment” or “an embodiment” or “an alternative embodiment” in different places in the present disclosure do not necessarily refer to the same embodiment. In addition, certain features, structures, or characteristics in one or more embodiments of the present disclosure may be suitably combined.

Furthermore, unless expressly stated in the claims, an order of the processing elements and sequences described herein, the use of numerical letters, or the use of other names are not intended to qualify the order of the processes and methods of the present disclosure. While some embodiments of the present disclosure that are currently considered useful are discussed in the foregoing disclosure by way of various examples, it should be appreciated that such details serve only illustrative purposes, and that additional claims are not limited to the disclosed embodiments. Rather, the claims are intended to cover all amendments and equivalent combinations that are consistent with the substance and scope of the embodiments of the present disclosure. For example, although the implementation of various components described above are embodied in a hardware device, it may also be implemented as a software only solution, e.g., an installation on an existing server or mobile device.

Similarly, it should be noted that in order to simplify the presentation of the disclosure of the present disclosure, and thereby aid in the understanding of one or more embodiments of the present disclosure, the foregoing descriptions of embodiments of the present disclosure sometimes group multiple features together in a single embodiment, accompanying drawings, or in a description thereof. However, the method of disclosure does not imply that more features are required for the objects of the present disclosure than are mentioned in the claims. Rather, the claimed subject matter may lie in less than all features of a single foregoing disclosed embodiment.

Some embodiments use counts to describe the count of components, attributes, and it should be understood that such counts used in the description of embodiments are modified in some examples by the modifiers “approximately,” “nearly,” or “substantially”. Unless otherwise noted, the terms “approximately,” “nearly,” or “substantially” indicates that a ±20% variation in the stated count is allowed. Correspondingly, in some embodiments, the numerical parameters used in the present disclosure and the claims are approximations, which change depending on the desired characteristics of individual embodiments. In some embodiments, the numerical parameters should consider the specified count of valid digits and use a general digit retention method. While the numerical domains and parameters used to confirm the breadth of their ranges in some embodiments of the present disclosure are approximations, in specific embodiments, such values are set to be as precise as possible within a feasible range.

For each of the patents, patent applications, patent application disclosures, and other materials cited in the present disclosure, such as articles, books, specification sheets, publications, documents, etc., are hereby incorporated by reference in their entirety into the present disclosure. Application history documents that are inconsistent with or conflict with the contents of the present disclosure are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those set forth herein, the descriptions, definitions and/or use of terms in the present disclosure shall prevail. Finally, it should be understood that the embodiments described herein are only used to illustrate the principles of the embodiments of the present disclosure. Other deformations may also fall within the scope of the present disclosure. As such, alternative configurations of embodiments of the present disclosure are viewed as consistent with the teachings of the present disclosure as an example, not as a limitation. Correspondingly, the embodiments of the present disclosure are not limited to the embodiments expressly presented and described herein.

1. A system, comprising an Emission Computed Tomography (ECT) scanner, wherein the ECT scanner comprises:
a detector defining an imaging field; and
a photon emission device disposed outside the imaging field and configured to emit photons before and during an ECT scan, wherein at least a portion of the photons enter the imaging field and strike the detector.

2. The system of claim 1, wherein the photon emission device is configured such that the photons irradiate a target region, and the target region covers the detector but does not cover a scanning object.

3. The system of claim 2, wherein the target region only covers the detector without covering any region outside the imaging field.

4. The system of claim 2, wherein the target region covers the detector and one or more regions outside the imaging field.

5. The system of claim 1, wherein an energy of the photons is in a range from 430 keV to 650 keV.

6. The system of claim 2, wherein the photon emission device comprises:
a photon emission source;
a first photon shielding structure disposed on a first side of the photon emission source facing away from the detector;
a second photon shielding structure disposed on a second side of the photon emission source facing the detector; and
a fixing structure that secures positions of the photon emission source and the second photon shielding structure relative to the first photon shielding structure, the fixing structure being made of a material that allows the photons to pass through.

7. The system of claim 6, wherein the target region is determined by adjusting a relative position of the photon emission source and the first photon shielding structure or a relative position of the first photon shielding structure and the second photon shielding structure.

8. The system of claim 6, wherein the photon emission source includes at least one of a positron-emitting radionuclide or a single-photon-emitting radionuclide.

9. The system of claim 6, wherein the first photon shielding structure includes a concave structure, and the photon emission source is secured within the concave structure.

10. The system of claim 9, wherein the photon emission source is secured to the bottom surface of the concave structure.

11. The system of claim 9, wherein the photon emission source is secured to be suspended in the concave structure.

12. The system of claim 9, wherein the second photon shielding structure is secured outside or within the concave structure and does not contact the bottom surface of the concave structure, and the second photon shielding structure is spaced apart from the photon emission source.

13. The system of claim 6, wherein the fixing structure comprises a first component surrounding the photon emission source, the first component is connected between the photon emission source and the first photon shielding structure, and the first component is connected between the second photon shielding structure and the first photon shielding structure.

14. The system of claim 13, wherein the fixing structure further comprises a second component connected between the photon emission source and the second photon shielding structure.

15. The system of claim 14, wherein the first component and the second component have different hardnesses.

16. The system of claim 1, wherein the photon emission device is arranged at a central axis of the imaging field and spaced apart from the detector along the central axis.

17. The system of claim 1, wherein the ECT scanner includes a pair of photon emission devices symmetrically arranged relative to a central axis of the imaging field, and the pair of photon emission devices are spaced apart from the detector along the central axis.

18-20. (canceled)

21. A method, the method being implemented on a computing device having at least one storage device and at least one processor, the method comprising:
obtaining first scan data collected by an Emission Computed Tomography (ECT) scanner in a blank scan without a scanning object in the imaging field, wherein the ECT scanner comprises a detector and a photon emission device, the detector defining an imaging field and collecting scan data, the photon emission device being disposed outside the imaging field and configured to emit photons before and during an ECT scan, wherein at least a portion of the photons enter the imaging field and strike the detector;
obtaining second scan data collected by the detector in the ECT scan with the scanning object in the imaging field; and
generating an attenuation-corrected ECT image of the scanning object based on the first scan data and the second scan data.

22. The method of claim 21, wherein the generating an attenuation-corrected ECT image of the scanning object based on the first scan data and the second scan data comprises:
determining first backscattering coincidence event data based on the first scan data;
determining radiological coincidence event data and second backscattering coincidence event data based on the second scan data; and
generating the attenuation-corrected ECT image of the scanning object based on the first backscattering coincidence event data, the second backscattering coincidence event data, and the radiological coincidence event data.

23. The method of claim 22, wherein the generating the attenuation-corrected ECT image of the scanning object based on the first backscattering coincidence event data, the second backscattering coincidence event data, and the radiological coincidence event data comprises:
determining reference blank scanning estimation for backscattering corresponding to the photon emission device based on the first backscattering coincidence event data, a scan duration of the blank scan, and a scan duration of the ECT scan; and
generating the attenuation-corrected ECT image of the scanning object based on the reference blank scanning estimation for backscattering, the second backscattering coincidence event data, and the radiological coincidence event data.

24-35. (canceled)
